(19) United States
(12) Patent Application Publication
(10) Pub. No.: US 2026/0334955 A1
(43) Pub. Date: Oct. 29, 2026

(54) OCCLUSIVE MEDICAL DEVICE

(71) Applicant: BOSTON SCIENTIFIC SCIMED, INC., MAPLE GROVE, MN (US)

(72) Inventors: Harishankar Natesan, Corcoran, MN (US); Jan-Hung Chen, Saint Paul, MN (US); Thyna M. Chau, Rochester, MN (US); Graham Krumpelmann, Stillwater, MN (US); Joseph C. Walker, Shoreview, MN (US); Mitul Ajit Shah, Maple Grove, MN (US); Rohith Jayaram, Maple Grove, MN (US); Joshua Mark Inouye, Brooklyn Park, MN (US); Dennis A. Peiffer, Brooklyn Park, MN (US); Brandom Alberto Figueroa Brenes, Plymouth, MN (US); Ellen Rose Zimmer, Medina, MN (US); Daniel L. Ross, Watertown, MN (US); Connor Thomas Gunsbury, Minneapolis, MN (US)

(73) Assignee: BOSTON SCIENTIFIC SCIMED, INC., MAPLE GROVE, MN (US)

(21) Appl. No.: 19/656,272

(22) Filed: Apr. 23, 2026

Related U.S. Application Data
(60) Provisional application No. 63/793,451, filed on Apr. 23, 2025.

(51) Int. Cl. A61B 17/12; A61B 17/00

(52) U.S. Cl. CPC A61B 17/12122; A61B 17/12172; A61B 2017/00243

(57) ABSTRACT

Medical devices including occlusive implants are disclosed. An example occlusive implant includes a distal hub, a proximal hub and an expandable framework including a plurality of struts spaced around a central longitudinal axis and forming a distal region, a proximal region and an engagement region positioned between the distal region and the proximal region. The proximal region includes a proximal first row of struts coupled to a row of first connecting nodes. The proximal first row of struts includes between 18 and 68 struts. The distal region includes a distal first row of struts coupled to a row of second connecting nodes. The distal first row of struts includes between 9 and 68 struts. The engagement region includes between 18 and 34 struts. Further, at least one node of the row of first connecting nodes includes an extrados region, an intrados region or both an extrados region and intrados region.

### CROSS REFERENCE TO RELATED APPLICATIONS

This application claims the benefit of priority of U.S. Provisional Application No. 63/793,451 filed Apr. 23, 2025, the entire disclosure of which is hereby incorporated by reference.

### TECHNICAL FIELD

The present disclosure pertains to medical devices and systems, and methods for manufacturing and using medical devices and systems. More particularly, the present disclosure pertains to medical implants for occluding a left atrial appendage.

### BACKGROUND

The left atrial appendage (LAA) is a small organ attached to the left atrium of the heart as a pouch-like extension. In patients suffering from atrial fibrillation, the left atrial appendage may not properly contract with the left atrium, causing stagnant blood to pool within its interior, which can lead to the undesirable formation of thrombi within the left atrial appendage. Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation are found in the left atrial appendage. As a treatment, medical devices have been developed which are positioned in the left atrial appendage and deployed to close off the ostium of the left atrial appendage. Over time, the exposed surface(s) spanning the ostium of the left atrial appendage becomes covered with tissue (a process called endothelization), effectively removing the left atrial appendage from the circulatory system and reducing or eliminating the number of thrombi which may enter the blood stream from the left atrial appendage. A continuing need exists for improved medical devices and methods to control thrombus formation within the left atrial appendage of patients suffering from atrial fibrillation.

### SUMMARY

An example occlusive implant includes a distal hub, a proximal hub and an expandable framework including a plurality of struts spaced around a central longitudinal axis of the occlusive implant and forming a distal region, a proximal region and an engagement region positioned between the distal region and the proximal region. Further, the plurality of the struts forming the proximal region includes a proximal first row of struts coupled to a row of first connecting nodes, and wherein the proximal first row of struts includes between 18 and 68 struts. Further, the plurality of the struts forming the distal region includes a distal first row of struts coupled to a row of second connecting nodes, and wherein the distal first row of struts includes between 9 and 68 struts. Further, the plurality of the struts forming the engagement region includes between 18 and 34 struts. Further, at least one node of the row of first connecting nodes includes an extrados region, an intrados region or both an extrados region and an intrados region.

In addition or alternatively to any example described herein, wherein at least one node of the row of second connecting nodes includes an extrados region, an intrados region or both an extrados region and an intrados region.

In addition or alternatively to any example described herein, wherein a strut of the plurality engagement struts is coupled to a fixation member at a fixation node, and wherein the fixation node includes an extrados region, an intrados region or both an extrados region and an intrados region.

In addition or alternatively to any example described herein, wherein the plurality of the struts forming the proximal region further includes a proximal second row of struts, and wherein the proximal second row of struts includes between 36 and 68 struts.

In addition or alternatively to any example described herein, wherein the plurality of the struts forming the distal region further includes a distal second row of struts, and wherein the distal second row of struts includes between 36 and 68 struts.

In addition or alternatively to any example described herein, wherein the plurality of the struts forming the proximal region further includes a proximal third row of struts, and wherein the proximal third row of struts includes between 36 and 68 struts.

In addition or alternatively to any example described herein, wherein the plurality of the struts forming the distal region further includes a distal third row of struts, and wherein the distal third row of struts includes between 36 and 68 struts.

In addition or alternatively to any example described herein, wherein the plurality of the struts forming the proximal region further includes a proximal fourth row of struts, and wherein the proximal fourth row of struts includes between 36 and 68 struts.

In addition or alternatively to any example described herein, wherein the plurality of the struts forming the distal region further includes a distal fourth row of struts, and wherein the distal fourth row of struts includes between 36 and 68 struts.

In addition or alternatively to any example described herein, wherein the plurality of the struts forming the proximal region further includes a proximal fifth row of struts, and wherein the proximal fifth row of struts includes between 36 and 68 struts.

In addition or alternatively to any example described herein, wherein the plurality of the struts forming the distal region further includes a distal fifth row of struts, and wherein the distal fifth row of struts includes between 36 and 68 struts.

In addition or alternatively to any example described herein, wherein a strut from the plurality of the struts forming the proximal region is connected to a strut from the plurality of the struts forming the engagement region at a proximal engagement node, and wherein the proximal engagement node includes an extrados region, an intrados region or both an extrados region and an intrados region.

In addition or alternatively to any example described herein, wherein a strut from the plurality of the struts forming the distal region is connected to a strut from the plurality of the struts forming the engagement region at a distal engagement node, and wherein the distal engagement node includes an extrados region, an intrados region or both an extrados region and an intrados region.

In addition or alternatively to any example described herein, wherein each strut of the proximal first row of struts and each strut of the distal first row of struts includes a width between 0.0025 inches and 0.020 inches and a thickness between 0.004 inches and 0.010 inches.

In addition or alternatively to any example described herein, wherein the ratio of the width to the thickness of each strut of the proximal first row of struts and each strut of the distal first row of struts is less than 1.0.

In addition or alternatively to any example described herein, wherein the proximal first row of struts includes a first strut and a second strut, and wherein the first strut and the second strut diverge from a node of the row of first connecting nodes to define an angle between 40 degrees and 80 degrees.

In addition or alternatively to any example described herein, further comprising an occlusive member disposed along an outer surface of the expandable framework.

An occlusive implant includes a distal hub, a proximal hub and an expandable framework including a plurality of struts spaced around a central longitudinal axis of the occlusive implant and forming a distal region. Further, the plurality of the struts forming the distal region includes a distal first row of struts coupled to a row of connecting nodes. Further, the row of connecting nodes includes a first connecting node, a second connecting node and a third connecting node. Further, the first connecting node has a first node length. Further, the second connecting node has a second node length the same as the first node length. Further, the third connecting node has a third node length different from the first node length. Further, the third connecting node is disposed between the first connecting node and the second connecting node.

In addition or alternatively to any example described herein, wherein at least one node of the row of connecting nodes includes an extrados region, an intrados region or both an extrados region and an intrados region

An occlusive implant includes an expandable framework configured to shift between a collapsed configuration and an expanded configuration. Further, the expandable framework includes a plurality of struts spaced around a central longitudinal axis of the expandable implant and forming a distal region, and a plurality of struts spaced around the central longitudinal axis of the expandable implant and forming a proximal region. Further, the plurality of struts forming the distal region are configured to shift between a collapsed configuration and an expanded configuration. Further, the plurality of struts forming the proximal region are configured to shift between a collapsed configuration and an expanded configuration. Further, the plurality of struts forming the distal region are configured to maintain the expanded configuration when the struts forming the proximal region are in a partially expanded configuration. Further, the plurality of struts forming the proximal region are configured to maintain the expanded configuration when the struts forming the distal region are in a partially expanded configuration.

### BRIEF DESCRIPTION OF THE DRAWINGS

The disclosure may be more completely understood in consideration of the following detailed description of various embodiments in connection with the accompanying drawings, in which:

FIG. 1 is a side view of an example occlusive implant;

FIG. 2 is a side view of the occlusive implant of FIG. 1 including an occlusive member;

FIG. 3 shows an example occlusive implant positioned in a heart;

FIG. 4 illustrates an occlusive implant positioned in a left atrial appendage;

FIG. 5 illustrates an occlusive implant positioned in a left atrial appendage;

FIG. 6 illustrates the occlusive implant of FIG. 1 in an unexpanded configuration;

FIG. 7 illustrates a node of the occlusive implant of FIG. 1;

FIG. 8A illustrates a portion of the occlusive implant of FIG. 1;

FIG. 8B illustrates a node of the occlusive implant of FIG. 1;

FIG. 9 illustrates a node of the occlusive implant of FIG. 1;

FIG. 10 illustrates a node of the occlusive implant of FIG. 1;

FIG. 11 illustrates a node of the occlusive implant of FIG. 1;

FIG. 12 illustrates a node of the occlusive implant of FIG. 1;

FIG. 13 illustrates a node of the occlusive implant of FIG. 1;

FIG. 14 illustrates a node of the occlusive implant of FIG. 1;

FIG. 15 illustrates a node of the occlusive implant of FIG. 1;

FIG. 16A illustrates a node of the occlusive implant of FIG. 1;

FIG. 16B illustrates a portion of an example occlusive implant in an unexpanded configuration;

FIG. 16C illustrates a portion of the occlusive implant of FIG. 16B in an expanded configuration;

FIG. 17 illustrates a node of the occlusive implant of FIG. 1;

FIG. 18 is a cross-sectional view of a strut of the occlusive implant of FIG. 6;

FIG. 19 is a cross-sectional view of a strut of the occlusive implant of FIG. 6;

FIG. 20A illustrates a view facing the proximal region of the occlusive implant of FIG. 1;

FIG. 20B is a detailed view of a portion of the proximal region of FIG. 20A;

FIG. 21A illustrates a view facing the distal region of the occlusive implant of FIG. 1;

FIG. 21B is a detailed view of a portion of the distal region of FIG. 21A;

FIG. 22 is a schematic side view of an example occlusive implant;

FIG. 23 is a schematic side view of an example occlusive implant;

FIG. 24 is a schematic side view of an example occlusive implant;

FIG. 25 is a schematic side view of an example occlusive implant;

FIG. 26 is a schematic side view of an example occlusive implant;

FIG. 27 is a schematic side view of an example occlusive implant;

FIG. 28 is a schematic side view of an example occlusive implant;

FIG. 29 is a schematic side view of an example occlusive implant.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed disclosure. Those skilled in the art will recognize that the various elements described and/or shown may be arranged in various combinations and configurations without departing from the scope of the disclosure. The detailed description and drawings illustrate example embodiments of the claimed disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The occurrence of thrombi in the left atrial appendage (LAA) during atrial fibrillation may be due to stagnancy of blood pooling in the LAA. The pooled blood may still be pulled out of the left atrium by the left ventricle, however less effectively due to the irregular contraction of the left atrium caused by atrial fibrillation. Therefore, instead of an active support of the blood flow by a contracting left atrium and left atrial appendage, filling of the left ventricle may depend primarily or solely on the suction effect created by the left ventricle. However, the contraction of the left atrial appendage may not be in sync with the cycle of the left ventricle. For example, contraction of the left atrial appendage may be out of phase up to 180 degrees with the left ventricle, which may create significant resistance to the desired flow of blood. Further still, most left atrial appendage geometries are complex and highly variable, with large irregular surface areas and a narrow ostium or opening compared to the depth of the left atrial appendage. These aspects as well as others, taken individually or in various combinations, may lead to high flow resistance of blood out of the left atrial appendage.

In an effort to reduce the occurrence of thrombi formation within the left atrial appendage and prevent thrombi from entering the blood stream from within the left atrial appendage, it may be desirable to develop medical devices and/or occlusive implants that close off the left atrial appendage from the heart and/or circulatory system, thereby lowering the risk of stroke due to thrombolytic material entering the blood stream from the left atrial appendage. Example medical devices and/or occlusive implants that close off the left atrial appendage are disclosed herein.

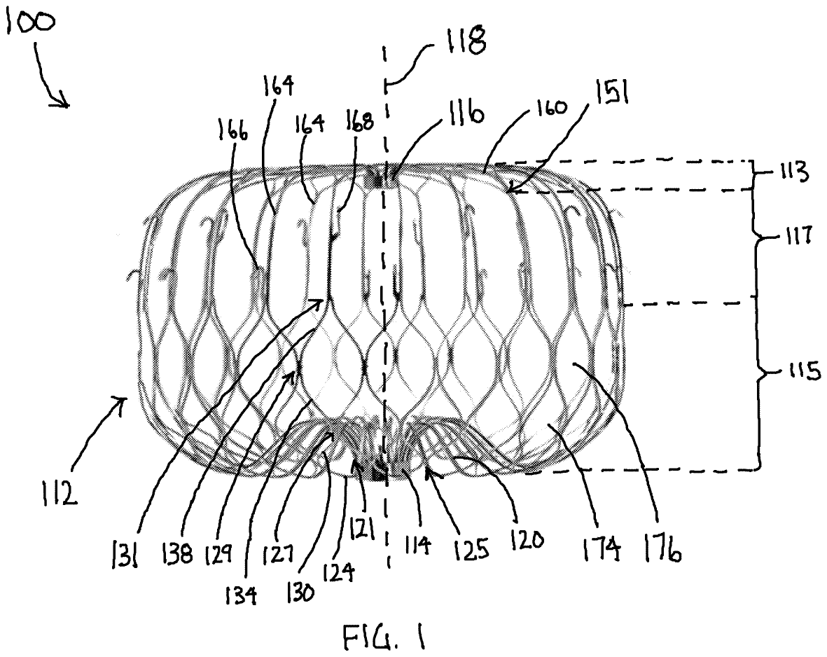

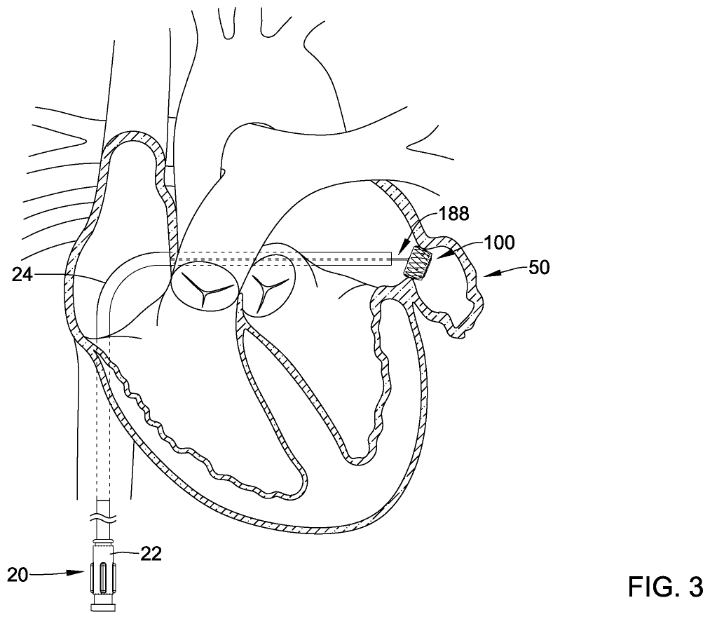

FIG. 1 illustrates an example occlusive implant 100. The occlusive implant 100 may include an expandable framework 112. The occlusive implant 100 may be configured to shift between a collapsed (e.g., delivery) configuration and an expanded (e.g., deployed) configuration. For example, the occlusive implant 100 may be in a collapsed configuration during delivery via an occlusion implant delivery system (an example of which is shown in FIG. 3), whereby the occlusive implant 100 expands to an expanded configuration once deployed from the occlusion implant delivery system.

Additionally, the expandable framework 112 may include a plurality of strut members (e.g., struts) forming a distal region 113. The expandable framework may also include a plurality of strut members forming a proximal region 115. The expandable framework may also include a plurality of strut members forming an engagement region 117. The strut members forming the distal region 113 may be interconnected with one another and extend circumferentially around a central longitudinal axis 118 to define the distal region 113 of the expandable framework 112. The strut members forming the proximal region 115 may be interconnected with one another and extend circumferentially around the longitudinal axis 118 to define the proximal region 115 of expandable framework 112. In some examples, the struts defining the distal region 113 may interconnect with the struts defining the engagement region 117. In some examples, the struts defining the proximal region 115 may interconnect the struts defining the engagement region 117. In some examples the distal region 113 may be considered the “leading” region of the expandable framework 112 as it enters into a left atrial appendage. In other examples, the proximal region 115 may be considered the leading region of the expandable framework 112 as it enters into the left atrial appendage.

FIG. 1 further illustrates that the proximal region 115 of the expandable framework 112 may include a plurality of struts 120 extending circumferentially around the longitudinal axis 118 of the expandable framework 112. FIG. 1 illustrates that that plurality of struts 120 may include one or more curved portions which are shaped such that they define a “recess” 121 extending distally into the expandable framework 112. As illustrated in FIG. 1, the recess 121 may extend circumferentially around the longitudinal axis 118. Further, FIG. 1 illustrates that each of the plurality of struts 120 may include a first end which is attached to a proximal hub 114. It can be appreciated that the proximal hub 114 may be aligned along the longitudinal axis 118 of the expandable framework 112. As will be described in greater detail below, FIG. 1 illustrates that the proximal hub 114 may be positioned such that it lies within the recess 121 defined by the plurality of struts 120.

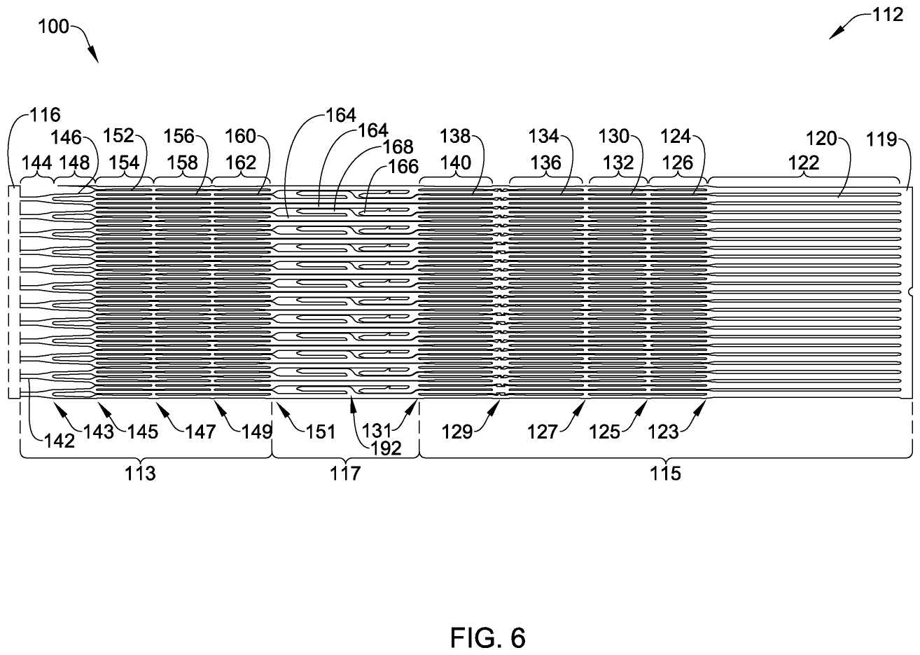

As will be described in greater detail herein, FIG. 1 illustrates a plurality of struts 124, 130, 134, 138 of the proximal region 115 and the struts 164 of the engagement region 117 interconnected with one another via nodes 125, 127, 129, 131 and extending circumferentially around the longitudinal axis 118. As will be described in greater detail herein, FIG. 1 also illustrates a plurality of struts 160, 164 of the distal region 113 and the struts 164 of the engagement region 117 interconnected with one another along via nodes 151 and extending circumferentially around the longitudinal axis 118. In some examples, each strut 164 of the engagement region 117 may extend from a distal engagement node 151 to a proximal engagement node 131. The struts forming the distal region 113, the proximal region 115, and the engagement region 117 are illustrated and further described with respect to FIG. 6 and FIGS. 20A-21B. As will be discussed in greater detail below, FIG. 1 illustrates that the struts 130 in combination with struts 134 may form a row of interstitial cells 174 and the struts 134 in combination with struts 138 may form a row of interstitial cells 176.

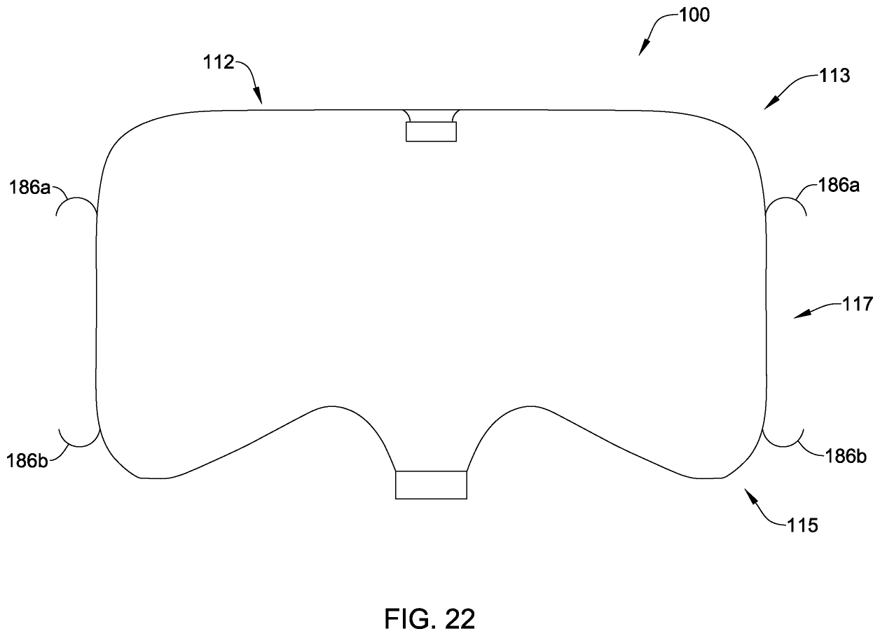

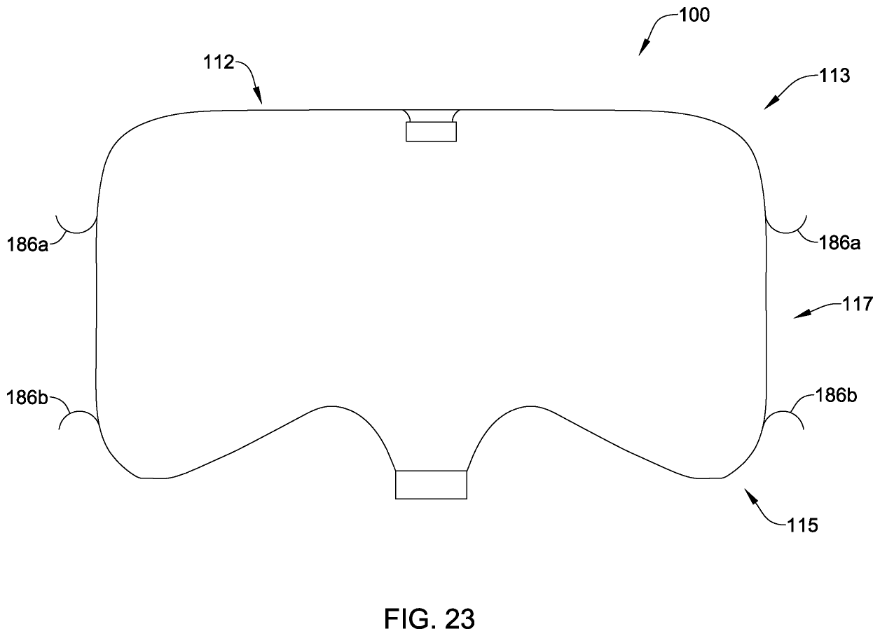

FIG. 1 further illustrates that the expandable framework 112 may include a plurality of fixation members 166, 168 disposed about a periphery of the expandable framework 112. For example, FIG. 1 shows that the fixation members 166, 168 may be disposed along one or more strut members 164 of the expandable framework 112. In other examples, any number of fixation members 166, 168 may be disposed along one or more strut members forming the distal region 113, the proximal region 115 and/or the engagement region 117. Some suitable, but non-limiting, examples of materials for the expandable framework 112 and/or the plurality of fixation members 166, 168 are disclosed herein. The plurality of fixation members 166, 168 may extend radially outward from the strut members of the expandable framework 112. In other words, the plurality of fixation members 166, 168 may extend radially away from the longitudinal axis 118 of the expandable framework 112. Further discussion about alternative configurations for fixation members along the expandable framework 112 is set forth herein with respect to FIGS. 22-23.

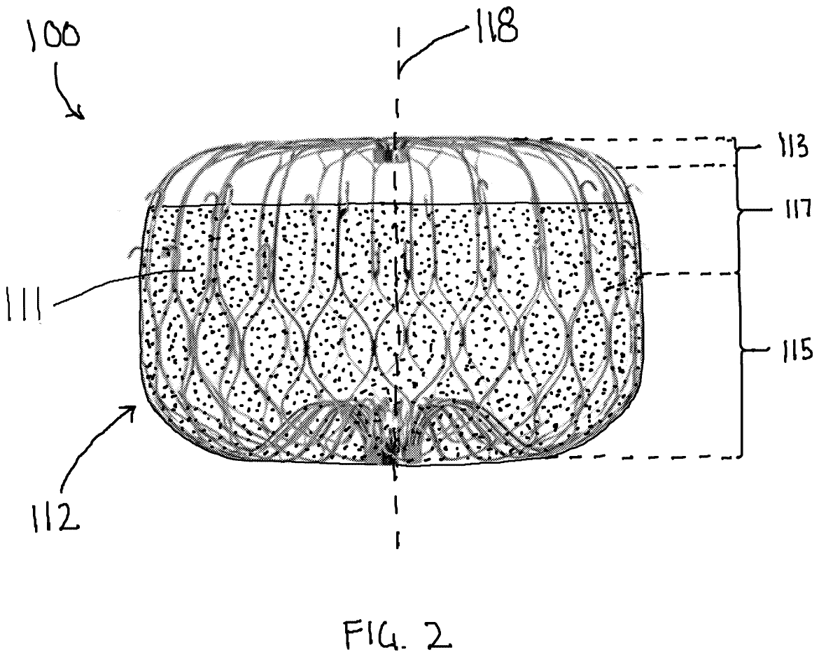

FIG. 2 illustrates that the occlusive implant 100 may also include an occlusive member 111 disposed on, disposed over, disposed about, or covering at least a portion of the expandable framework 112. For example, FIG. 2 illustrates that the occlusive implant 100 may include an occlusive member 111 which may be disposed on, disposed over, disposed about or cover at least a portion of an outer (or outwardly facing) surface of the expandable framework 112, whereby the occlusive member 111 may be permeable or impermeable to blood and/or other fluids, such as water. In some embodiments, the occlusive implant 100 may include an occlusive member 111 which includes a woven, braided and/or knitted material, a fiber, a sheet-like material, a fabric, a polymeric membrane, a metallic or polymeric mesh, a porous filter-like material, or other suitable construction.

FIG. 3 illustrates that the occlusive implant 100 inserted and advanced through a body lumen via an occlusive implant delivery system 20. FIG. 3 illustrates the occlusive implant 100 being delivered and positioned within a left atrial appendage 50. In some instances, an occlusive implant delivery system 20 may include a delivery catheter 24 which is guided toward the left atrium via various chambers and lumens of the heart (e.g., the inferior vena cava, the right atrium, etc.) to a position adjacent the left atrial appendage 50.

The delivery system 20 may include a hub member 22 coupled to a proximal region of the delivery catheter 24. The hub member 22 may be manipulated by a clinician to direct the distal end region of the delivery catheter 24 to a position adjacent the left atrial appendage 50. In some embodiments, an occlusive implant delivery system may include a core wire 188. Further, a proximal region of the expandable framework 112 (shown in FIG. 1) may be configured to releasably attach, join, couple, engage, or otherwise connect to the distal end of the core wire 188. In some embodiments, an end region (typically at the proximal region 115) of the expandable framework 112 may include a threaded insert coupled thereto, for example, at the proximal hub 114 (shown in FIG. 1). In some embodiments, the threaded insert may be configured to and/or adapted to couple with, join to, mate with, or otherwise engage a threaded member disposed at the distal end of a core wire 188. Other means of releasably coupling and/or engaging the proximal region of the expandable framework 112 to the distal end of the core wire 188 are also contemplated.

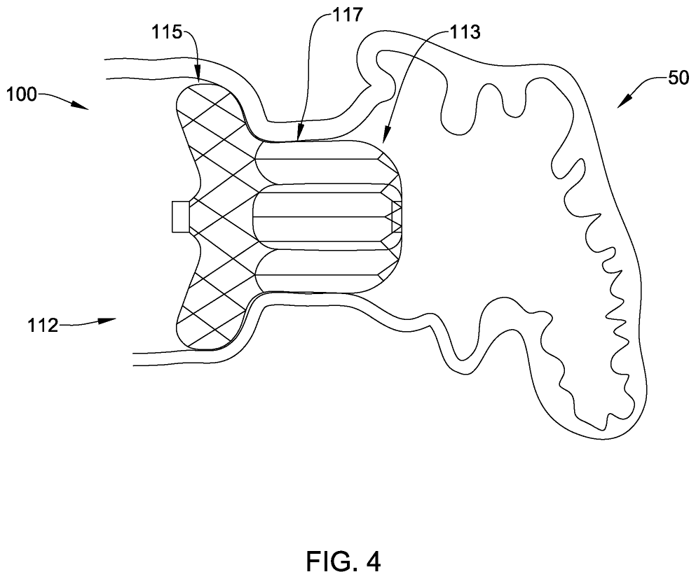

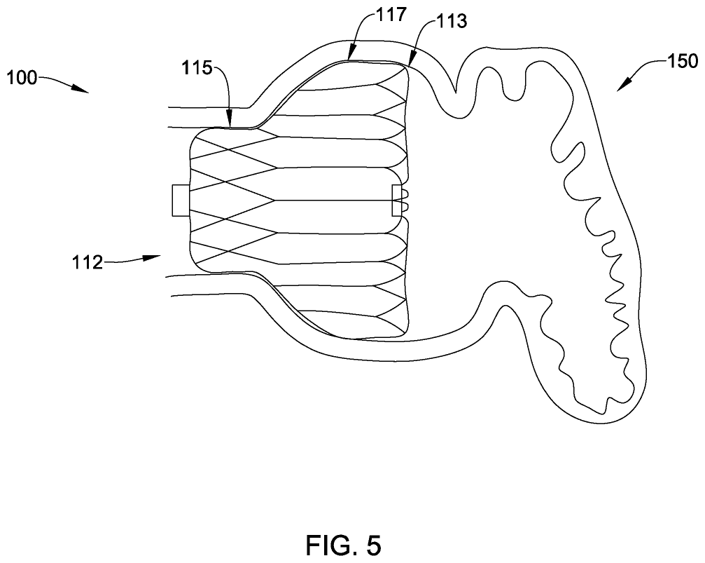

It can be appreciated that it may be desirable to configure the occlusive implant 100 disclosed herein to substantially conform (e.g., adapt) to and/or be in sealing engagement with the shape and/or geometry of the lateral walls of a left atrial appendage in the expanded configuration. For example, FIGS. 4-5 and the corresponding discussion set forth herein illustrates that the occlusive implant 100 may expand to a size, extent, or shape less than or different from a maximum unconstrained extent, as determined by the surrounding tissue and/or lateral wall of the left atrial appendage 50. It can be appreciated that elements of the distal region 113, the proximal region 115 and the engagement region 117 of the expandable framework 112 may be tailored to increase the flexibility, compliance and radial strength of the expandable framework 112 while minimizing structural fatigue (e.g., radial strain forces along one or more struts or node regions), thereby permitting the expandable framework 112 to conform to the tissue around it, rather than forcing the tissue to the occlusive implant 100.

It can be appreciated that a plurality of elements of the expandable framework 112 may together influence the compliance, flexibility and radial strength of the distal region 113, the proximal region 115 and the engagement region 117 of the expandable framework 112, thereby allowing the expandable framework 112 to substantially conform to and/or be in sealing engagement with the lateral walls of variously-shaped left atrial appendages while maintaining sufficient radial strength for optimizing sealing capabilities.

Depending on the specific anatomical geometry in which the occlusive implant 100 is deployed, various regions of expandable framework 112 (e.g., distal region 113, the proximal region 115 and the engagement region 117) may be required to expand to a size, extent, or shape less than or different from a maximum unconstrained extent of that respective region, as determined by the surrounding tissue and/or the lateral walls of the left atrial appendage 50. Accordingly, it may be desirable to configure one or more regions of the expandable framework 112 to be able to maintain a constrained configuration while other regions of the expandable framework 112 expand into a maximum (or near-maximum) expanded configuration. For example, FIG. 4 illustrates that it may be beneficial to configure the expandable framework 112 such that the plurality of struts defining the proximal region 115 may maintain an expanded, fully deployed configuration (e.g., deployed to its fully expanded diameter) despite at least a portion of the distal region 113, the engagement region 117 or portions of both the distal region 113 and the engagement region 117 being held in a compressed configuration (e.g., partially expanded, not fully expanded, etc.). Additionally, FIG. 5 illustrates that it may also be beneficial to configure the expandable framework 112 such that that the plurality of struts defining the distal region 113 may maintain an expanded, fully deployed configuration (e.g., deployed to its fully deployed diameter) despite at least a portion of the proximal region 115, the engagement region 117 or portions of both the proximal region 115 and the engagement region 117 being held in a compressed configuration (e.g., partially expanded, not fully expanded, etc.) whereby the proximal region 115 has been compressed (or maintained in a constrained configuration) to a diameter (e.g., width) smaller than its fully deployed (e.g., fully expanded) diameter.

In other words, it may be desirable to configure the expandable framework 112 to include one or more elements which together permit compressive forces applied to the struts defining distal region 113 to be dissociated from the struts defining proximal region 115, thereby permitting the proximal region 115 to maintain a generally expanded configuration while the distal region 113 is compressed. Similarly, it may be desirable to configure the expandable framework 112 to include one or more elements which together permit compressive forces applied to the struts defining the proximal region 115 to be dissociated from the struts defining distal region 113, thereby permitting the distal region 113 to maintain a generally expanded configuration while the proximal region 115 is compressed. In other words, it may be desirable to configure the expandable framework 112 to include one or more elements which together disperse forces applied to the struts defining the distal region 113 such that the compressive forces are limited from translating through the struts defining the engagement region 117 and effecting the ability of the struts defining the proximal region 115 to maintain an expanded configuration. Likewise, it may be desirable to configure the expandable framework 112 to include one or more elements which together disperse forces applied to the struts defining the proximal region 115 such that the compressive forces are limited from translating through the struts defining the engagement region 117 and effecting the struts defining the distal region 113 to maintain an expanded configuration.

Elements of the expandable framework 112 that together may permit compressive forces applied to the struts defining the distal region 113 and proximal region 115 to be dissociated from the struts defining proximal region 115 and distal region 113, respectively, may include the thickness of the strut members of the distal region 113, the proximal region 115 and/or the engagement region 117. Further, other elements may include the total number of struts within the proximal region 115 and/or the total number of struts within the distal region 113, the total number of struts within a given row of struts as defined herein, and the number of struts positioned within a proximal and/or a distal path lengths as defined herein. Further yet, other elements may include the geometry of various nodes of the expandable framework 112, wherein one or more node geometries may include extrados or intrados material positioned along specific regions of one or more struts and/or regions where one or more struts are joined. Further yet, other elements may include the configuration of an interior angle of the distal shoulder of the expandable framework 112 and/or the interior angle of the proximal shoulder of the expandable framework 112. Further yet, other elements may include the configuration of one or more angles between adjacent struts forming one or more individual cells of the expandable framework 112. It can be appreciated that any of these individual elements may be tailored alone or in combination with any other elements disclosed herein to control one or more mechanical properties (e.g., flexibility, compliance, radial strength, hoop stress, etc.) of the expandable framework 112 while minimizing structural fatigue and allowing the dissociation of compressive forces between the distal region 113 and the proximal region 115 as described herein.

As discussed herein, FIG. 4 illustrates the occlusive implant 100 deployed within an example left atrial appendage 50. It can be appreciated from the discussion herein that the deployed configuration of the occlusive implant 100 may include a configuration in which the occlusive implant 100 has been deployed from the delivery catheter 24 (shown in FIG. 3) and has been expanded to be positioned adjacent the lateral walls defining a portion of the left atrial appendage 50.

FIG. 4 illustrates the framework 112 in an expanded configuration whereby the proximal region 115 of the expandable framework 112 has expanded radially outward to its fully expanded configuration despite the distal region 113 being held in a compressed configuration by the lateral walls of the left atrial appendage 150. Further, the proximal region 115 of the expandable framework 112 has expanded radially outward beyond the diameter of the distal region 113 of the expandable framework 112 such that at least a portion of the engagement region 117 and/or the proximal region 115 extends along the lateral wall of the left atrial appendage 150, thereby enhancing the sealing capabilities of the occlusive implant 100 along the lateral wall of the left atrial appendage 150.

As discussed herein, FIG. 5 illustrates the occlusive implant 100 deployed within another example left atrial appendage 150. It can be appreciated from the discussion herein that the deployed configuration of the occlusive implant 100 may include a configuration in which the occlusive implant 100 has been deployed from the delivery catheter 24 (shown in FIG. 3) and has been expanded to be positioned adjacent the lateral walls defining a portion of the left atrial appendage 150.

FIG. 5 illustrates the framework 112 in an expanded configuration whereby the distal region 113 of the expandable framework 112 has expanded radially outward to its fully expanded configuration despite the proximal region 115 being held in a compressed configuration by the lateral walls of the left atrial appendage 150. Further, the distal region 113 of the expandable framework 112 has expanded radially outward beyond the diameter of the proximal region 115 of the expandable framework 112 such that at least a portion of the engagement region 117 and/or the distal region 113 extends along the lateral wall of the left atrial appendage 150, thereby enhancing the sealing capabilities of the occlusive implant 100 along the lateral wall of the left atrial appendage 150.

In some examples, the expandable framework 112 shown in FIG. 1 may be manufactured by machining (e.g., laser cutting) a unitary tubular member into a geometric pattern and then expanding the unitary tubular member to form the expandable framework 112 illustrated in FIG. 1. FIG. 6 illustrates a flat, unexpanded view of a geometric pattern which may be machined into a unitary tubular member which may then be expanded into the expandable framework 112 shown in FIG. 1.

Additionally, it can be appreciated that utilizing a particular manufacturing methodology (e.g., laser cutting) may permit the expandable framework 112 to include a variety of geometric configurations. As will be discussed in greater detail herein, utilizing a particular manufacturing methodology (e.g., laser cutting) may permit the framework 112 (including the plurality of struts forming the distal region 113, the plurality of struts forming the proximal region 115 and the plurality of struts forming the engagement region 117) to include features (e.g., geometry, arrangement, strut thickness, node shape, etc.) that allow the proximal region 115 of the expandable framework 112 to maintain an expanded, fully deployed configuration (e.g., deployed to its fully expanded diameter) despite the distal region 113 being held in a compressed configuration and also permit the distal region 113 of the expandable framework 112 to maintain an expanded, fully deployed configuration (e.g., deployed to its fully expanded diameter) despite the proximal region 115 being held in a compressed configuration.

FIG. 6 illustrates the plurality of interconnected strut members forming the proximal region 115, whereby the plurality of interconnected strut members extend away from a proximal hub attachment region 119. It can be appreciated that the proximal hub attachment region 119 may define the portion of the unitary tubular member in which the proximal hub 114 (shown in FIG. 1) may be attached in a subsequent manufacturing step. FIG. 6 illustrates that the proximal region 115 may include a row of proximal engagement nodes 131 separating the engagement region 117 from the proximal region 115. Further, FIG. 6 illustrates that the proximal region 115 may include a plurality of rows (e.g., a plurality of circumferential rows) of struts extending between the proximal engagement nodes 131 and the proximal hub attachment region 119. For example, FIG. 6 illustrates that the proximal region 115 may include a row of proximal hub connecting struts 122 extending from the proximal hub 114 to a row of first connecting nodes 123. Further, FIG. 6 illustrates that the proximal region 115 may further include a first row of struts 126, a second row of struts 132, a third row of struts 136 and a fourth row of struts 140 positioned between the row of first connecting nodes 123 and the row of proximal engagement nodes 131.

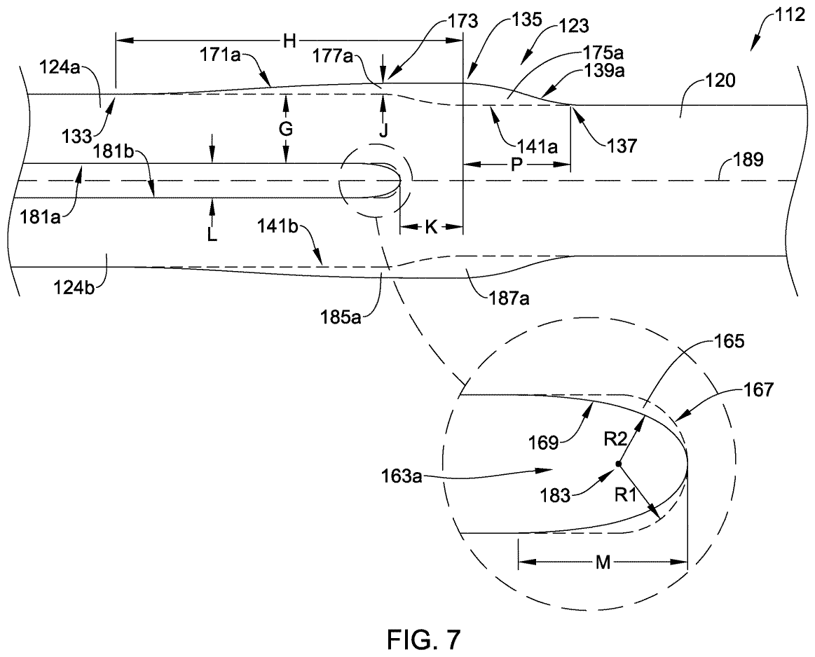

Further, FIG. 6 and FIG. 7 illustrate that a strut 120 of the row of proximal hub connecting struts 122 may be connected to a first strut 124a and a second strut 124b of the first row of struts 126 at a first connecting node 123. The node 123 may be defined as the general region of the expandable framework 112 in which the ends of the struts 124a, 124b, 120 are joined together. It can be appreciated that the geometric configuration of one or more nodes (e.g., node 123) of the expandable framework 112 may include one or more features which may increase the radial expansion forces of the expandable framework 112 while reducing the structural fatigue of the struts (e.g., struts 124a, 124b, 120) that are joined together at a given node (e.g., node 123). For example, the geometric configuration of the node 123 may be configured to lower the strain (e.g., circumferential strain, radial strain) in the struts 120, 124a, 124b which are joined together at the node 123.

FIG. 7 illustrates the node 123 as viewed on the flattened projection of the expandable framework 112 of FIG. 6. Further, for reference and discussion purposes, FIG. 7 includes a phantom line 141a which may represents a “base node” geometric configuration. The base node configuration represented by the phantom line 141a will be used as a reference geometry when describing and defining the geometric features of the node 123 which increase the radial expansion forces of the expandable framework 112 while reducing the structural fatigue of the nodes (e.g., node 123) and the struts (e.g., struts 124a, 124b, 120) that are joined at a given node (e.g., node 123). Additionally, for simplicity purposes, the following description describes the geometric configuration of the node 123 along one edge of the node 123. However, it can be appreciated that the node 123 geometry described herein is also present along the opposite edge of the node 123 given that the node 123 is symmetrical along its longitudinal axis 189.

FIG. 7 illustrates that the node 123 may include a first extrados region 177a which extends along an extrados length “H” from a first end position 133 along the outer surface of the expandable framework 112 shown in FIG. 7 to a second end position 135 along the outer surface of the expandable framework 112 shown in FIG. 7. In some examples, the extrados length H may be about 0.0200 inches to about 0.0350 inches, or about 0.0225 inches to about 0.0325 inches, or about 0.0250 inches to about 0.0300 inches, or about 0.0275 inches.

It can further be appreciated that the extrados region 177a may taper from the first end position 133 along the strut 124a to a maximum width (e.g., lateral extent) at a position 135 along the outer surface 171a of the expandable framework 112.

It can be further appreciated that this extrados region 177a may include strut and/or node material which extends beyond the bound of the base strut (described above) defined by the phantom line 141a. For example, FIG. 7 illustrates that the extrados region 177a may include an extrados width “J” of material extending from the phantom line 141a to the position 173 along the outer surface 171a of the expandable framework 112. In some examples, the extrados width J may be about 0.0006 inches to about 0.0012 inches, or about 0.0008 inches to about 0.0010 inches, or about 0.0009 inches.

FIG. 7 further illustrates that the node 123 may include a second extrados region which includes a transition extrados line 139a extending along an extrados length “P” from the second end position 135 along the outer surface of the expandable framework 112 shown in FIG. 7 to a transition end position 137 along the outer surface of the expandable framework 112. In some examples, the extrados length P may be about 0.00085 inches to about 0.075 inches, or about 0.001 inches to about 0.050 inches, or about 0.0025 inches to about 0.050 inches, or about 0.005 inches to about 0.025 inches.

FIG. 7 illustrates that the transition extrados line 139a may taper along from the second end position 135 to the transition end position 137 located along the strut 120. It can be appreciated the node 123 may include a transition volume of extrados material 175a which may be defined as a volume of node extrados material positioned between the phantom line 141a and the transition extrados line 139a and extending from the second end position 135 to the transition end position 137.

The detailed view of FIG. 7 further illustrates an inner node region 163a. The inner node region 163a may be defined as the region in which an inner surface of 181a of the strut 124a transitions to the inner surface 181b of the strut 124b along a curved inner surface 169. Similar to the phantom line 141a described herein, for reference and discussion purposes, FIG. 7 includes a phantom line 167 which may represents an inner base node geometric configuration. The inner base node configuration represented by the phantom line 167 will be used as a reference geometry when describing and defining the geometric features of the inner node region 163a which increase the radial expansion forces of the expandable framework 112 while reducing the structural fatigue of the nodes (e.g., node 123) and the struts that are joined at a given node (e.g., node 123).

The detailed view of FIG. 7 illustrates that the inner node region 163a may include strut and/or node material which extends beyond the bound of the base strut defined by the phantom line 167. For purposes of discussion herein, the inner node material extending between the phantom line 167 and the curved inner surface 169 may be referred to as an intrados region 165. FIG. 7 illustrates that the intrados region 165 may include an intrados length “M” which may be about 0.0020 inches to about 0.0060 inches, or about 0.0030 inches to about 0.0050 inches, or about 0.0035 inches to about 0.0045 inches, or about 0.0038 inches.

FIG. 7 illustrates that, for reference purposes, the phantom line 167 may be defined by an inner radius R₁. The inner radius R₁ may be a uniform (e.g., constant) radius whereby the distance from the center point 183 to any point on the phantom line 167 is the same along the curved portion of the phantom line 167. Further, FIG. 7 illustrates that the curved inner surface 169 of the inner node region 163a may include a variable inner radius R₂ whereby the distance from the center point 183 to any point on the curved inner surface 169 changes along the curved portion inner surface 169. It can be appreciated that, in some examples, the uniform inner radius R₁ may define a generally U-shaped phantom line 167. In contrast, FIG. 7 illustrates that the variable radius R₂ may define a generally ovular-shaped curved inner surface 169 as compared to the generally U-shaped phantom line 167. It can be appreciated that both the additional material defining the intrados region 165 in combination with the variable radius configuration of the inner node region 163a may may increase the radial expansion forces of the expandable framework 112 while reducing the structural fatigue of the struts (e.g., struts 124a, 124b, 120) that are joined together at a given nodal region (e.g., node 123).

FIG. 7 further illustrates that the node 123 may include an extrados extension distance “K” which may be defined as the distance from the apex of the curved inner surface 169 to the second end position 135. In some examples, the extrados extension distance K may be about 0.0020 inches to about 0.0070 inches, or about 0.0030 inches to about 0.0060 inches, or about 0.0040 inches to about 0.0050 inches, or about 0.0045 inches.

FIG. 7 further illustrates that each strut 124a, 124b may include a base strut width “G” which may be defined as the width of each strut 124a, 124b extending from the inner surfaces inner surface of 181a, 181b of each strut 124a, 124b, respectively, to the phantom lines 141a, 141b of each strut 124a, 124b. In some examples, the base strut width G may be about 0.0030 inches to about 0.0080 inches, or about 0.0040 inches to about 0.0070 inches, or about 0.0050 inches to about 0.0060 inches, or about 0.0056 inches.

FIG. 7 further illustrates that expandable framework 112 may include a strut-to-strut inner gap distance “L” which may be defined as the distance between the inner surfaces inner surface of 181a, 181b of each strut 124a, 124b, respectively. In some examples, the strut-to-strut inner gap distance L may be about 0.0010 inches to about 0.0040 inches, or about 0.0015 inches to about 0.0035 inches, or about 0.0020 inches to about 0.0030 inches, or about 0.0028 inches.

In some examples, the ratio of the intrados length M to the strut-to-strut inner gap distance L may be about 1 to about 3, or about 1.25 to about 2, or about 1.30 to about 1.5, or about 1.35.

In some examples, the ratio of the extrados width J to the base strut width G may be about 0.03 to about 0.30, or about 0.10 to about 0.20, or about 0.14 to about 0.18, or about 0.16.

In some examples, the ratio of the extrados length H to the extrados width J may be about 10 to about 200, or about 15 to about 100, or about 20 to about 75, or about 25 to about 50, or about 30.6.

In some examples, the ratio of the extrados extension distance K to the base strut width G may be about 0 to about 2, or about 0.25 to about 1.5, or about 0.50 to about 1, or about 0.80.

As discussed herein, the node 123 may include an extrados region 185a and a transition volume of material 187a which may include all the features described herein with respect to the extrados region 177a and transition volume of extrados material 175a. It can be appreciated that extrados region 185a and transition volume of material 187a may be mirrored (e.g., symmetrical) geometries of the extrados region 177a and transition volume of extrados material 175a taken along the longitudinal axis 189 of the node 123.

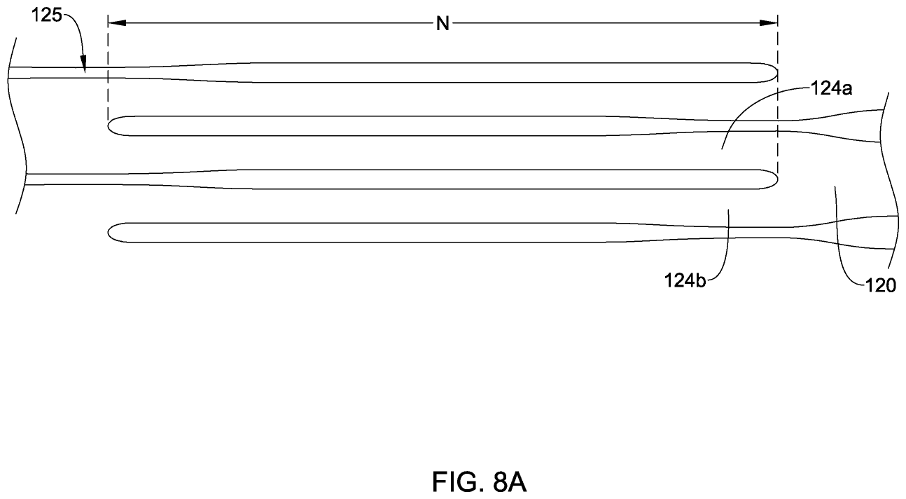

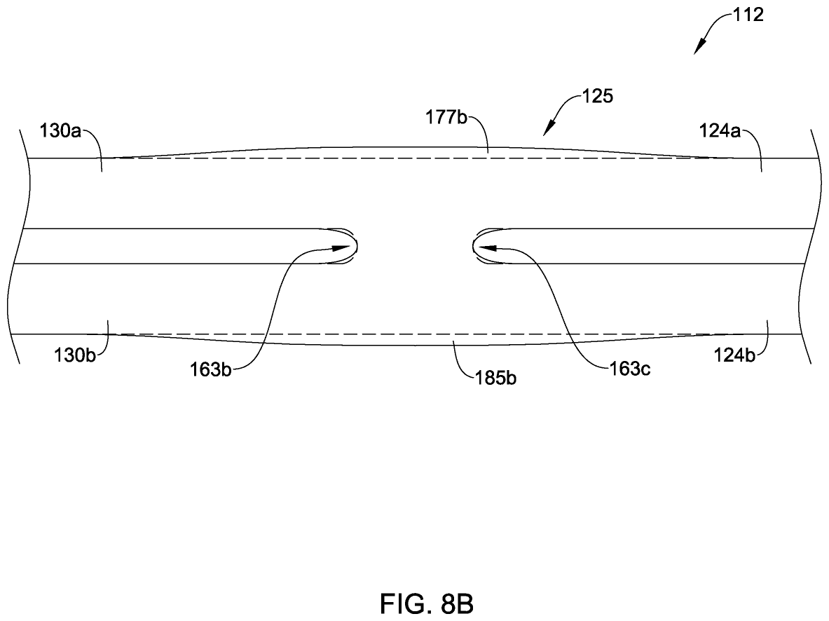

Further, FIG. 8A illustrates that the struts 124a, 124b may have a strut length “N”. In some examples, the strut length N may be about may be about 0.005 inches to about 0.250 inches, or about 0.010 inches to about 0.250 inches, or about 0.025 inches to about 0.225 inches, or about 0.050 inches to about 0.200 inches. Further, In some examples, the ratio of the extrados length H (shown in FIG. 7) to the strut length N may be about 0.10 to about 0.50, or about 0.15 to about 0.45, or about 0.20 to about 0.40, or about 0.25 to about 0.30, or about 0.29.

FIG. 6 and FIG. 8B illustrate that the first strut 124a and the second strut 124b of the first row of struts 126 may be connected to a first strut 130a and a second strut 130b of the second row of struts 132 at a second connecting node 125. It can be appreciated that the geometry of the node 125 may include extrados material 177b, 185b and inner node regions 163b, 163c which may be similar in form and function to the extrados region 177a and inner node region 163a described herein. It can be further appreciated that the ratios of the geometric relationships describing the extrados and intrados regions of node 123 may also apply to the node 125.

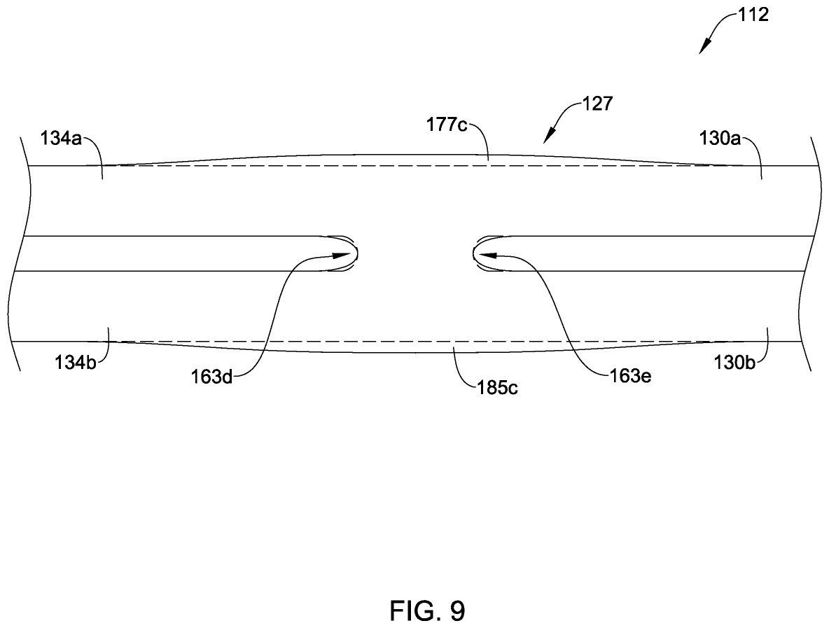

FIG. 6 and FIG. 9 illustrate that the first strut 130a and the second strut 130b of the second row of struts 126 may be connected to a first strut 134a and a second strut 134b of the third row of struts 132 at a third connecting node 127. It can be appreciated that the geometry of the node 127 may include extrados material 177c, 185c and inner node regions 163d, 163e which may be similar in form and function to the extrados region 177a and inner node region 163a described herein. It can be further appreciated that the ratios of the geometric relationships describing the extrados and intrados regions of node 123 may also apply to the node 127.

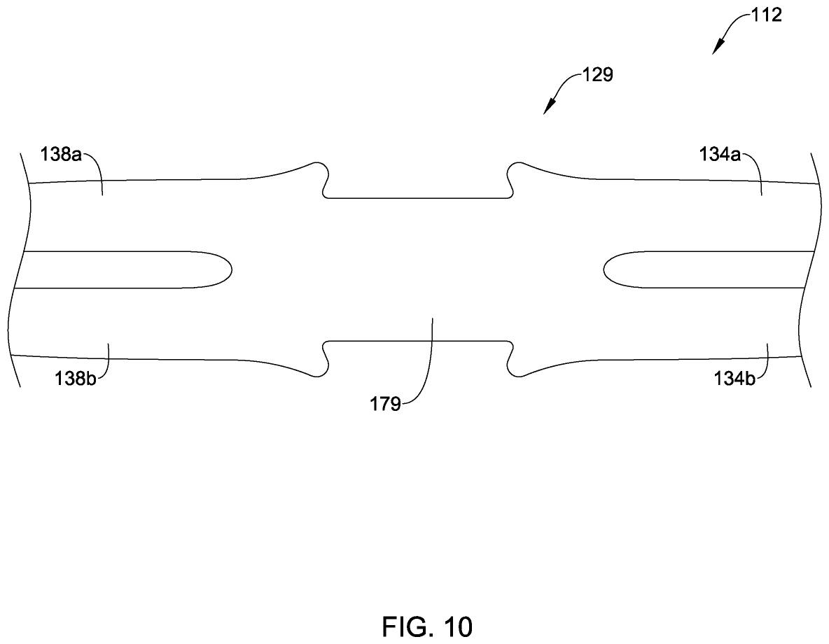

Further, FIG. 6 and FIG. 10 illustrate that the first strut 134a and the second strut 134b of the third row of struts 136 may be connected to a first strut 138a and a second strut 138b of the fourth row of struts 140 at a fourth connecting node 129. It can be appreciated that the node 129 may include a marker band attachment region 179 which may be configured to accept a radiopaque marker band during manufacturing of the occlusive implant 100.

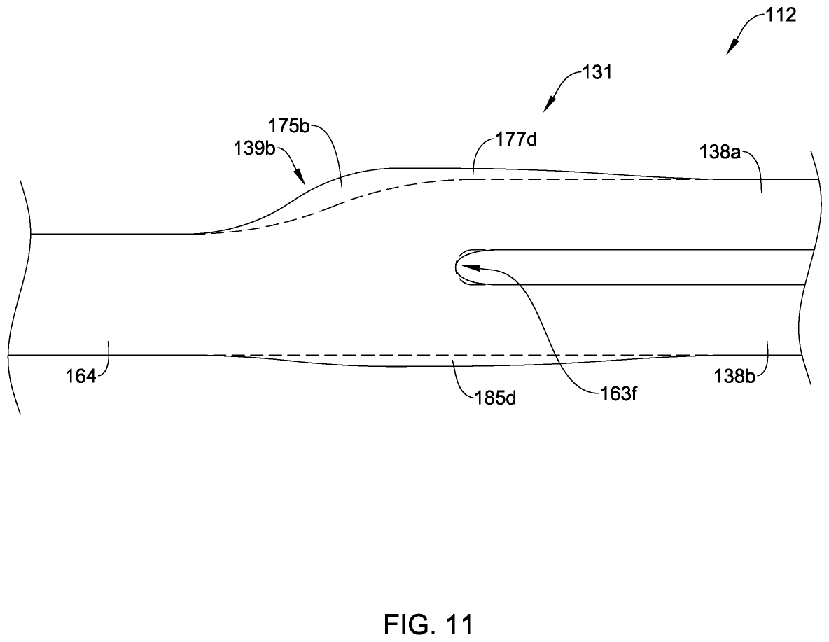

Further, FIG. 6 and FIG. 11 illustrate that the first strut 138a and the second strut 138b of the fourth row of struts 140 may be connected to a strut 164 of the engagement region 117 at a proximal engagement node 131. It can be appreciated that the geometry of the node 131 may include extrados material 177d, 185d and inner node region 163f which may be similar in form and function to the extrados region 177a and inner node region 163a described herein. It can be further appreciated that the ratios of the geometric relationships describing the extrados and intrados regions of node 123 may also apply to the node 131. Further, FIG. 11 illustrates that the node 131 may include a transition extrados line 139b which may include a similar but slightly modified shape when compared to the transition extrados line 139a described herein. For example, the transition extrados line 139b may be configured such that it defines a relatively greater transition volume of extrados material 175b compared to the transition volume of extrados material 175a described herein.

It can be appreciated that the number of the struts 120 of the row of proximal hub connecting struts 122 may include 18 struts to 34 struts, the number of the struts 124 of the first row of struts 126 may include 36 struts to 68 struts, the number of the struts 130 of the second row of struts 132 may include 36 struts to 68 struts, the number of the struts 134 of the third row of struts 136 may include 36 struts to 68 struts and the number of the struts 138 of the fourth row of struts 140 may include 36 struts to 68 struts.

Further, FIG. 6 illustrates that the lengths of the struts 120 of the row of proximal hub connecting struts 122 may be longer than the struts 124 of the first row of struts 126, the struts 130 of the second row of struts 132, the struts 134 of the third row of struts 136, and the struts 138 of the fourth row of struts 140. Further, the struts 124 of the first row of struts may be about the same length as the struts 130 of the second row of struts. Further, the struts 134 of the third row of struts 136 may be about same length as the struts 138 of the fourth row of struts 140. Further, the struts 134 of the third row of struts 136 and the struts 138 of the fourth row of struts 140 may be shorter than the struts 120 and longer than the struts 124 of the first row of struts 126 and the struts 130 of the second row of struts 132.

In some examples, the length of the struts 120 of the row of proximal hub connecting struts 122 may be about 0.25 inches to about 0.60 inches. In some examples, the length of the struts 124 of the first row of struts 126 may be about 0.060 inches to about 0.250 inches. In some examples, the length of the struts 130 of the second row of struts 132 may be about 0.060 inches to about 0.250 inches. In some examples, the length of the struts 134 of the third row of struts 136 may be about 0.060 inches to about 0.250 inches. In some examples, the length of the struts 138 of the fourth row of struts 140 may be about 0.060 inches to about 0.250 inches.

Further, it can be appreciated that while FIG. 6 illustrates that the proximal region 115 of the occlusive implant 100 includes four rows of struts 124, 130, 134, 138 positioned between the row of first connecting nodes 123 and the row of proximal engagement nodes 131, other configurations are contemplated. For example, the occlusive implant 100 may include 1, 2, 3, 4, 5, 6, 7, 8 or more rows of struts positioned between the row of first connecting nodes 123 and the row of proximal engagement nodes 131.

Further, it can be appreciated that while FIG. 6 illustrates that the proximal region 115 of the occlusive implant 100 includes five rows of struts 122, 144, 148, 154, 158, 162 positioned between the proximal hub 114 and the row of proximal engagement nodes 131, other variations are contemplated. For example, the occlusive implant 100 may include 1, 2, 3, 4, 5, 6, 7, 8 or more rows of struts positioned between the proximal hub 114 and the row of proximal engagement nodes 131.

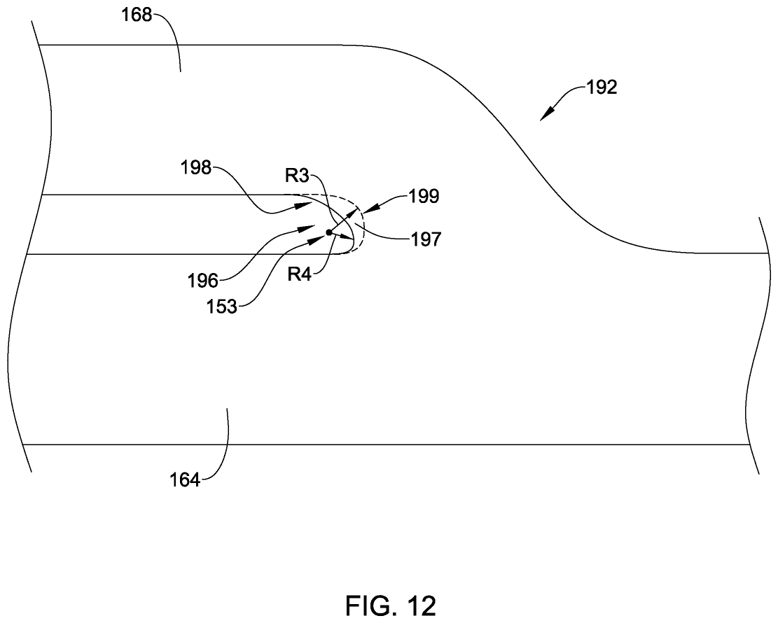

FIG. 6 and FIG. 12 illustrate that the fixation member 168 may be connected to a strut 164 of the engagement region 117 at a node 192 (e.g., fixation node 192). The node 196 may include an inner node region 196. Similar to the phantom line 141a described herein, for reference and discussion purposes, FIG. 12 includes a phantom line 199 which may represent an inner base node geometric configuration. The inner base node configuration represented by the phantom line 199 will be used as a reference geometry when describing and defining the geometric features of the inner node region 196. It can be appreciated that the discussion herein with respect to the fixation member 168, node 192 and the strut 164 may also describe the arrangement and geometric configuration of the fixation member 166 coupled to the strut 164. For example, the fixation member 166 may be connected to a strut 164 at a node which is similar in form and function to the node 192 described herein.

FIG. 12 illustrates that the inner node region 196 may include strut and/or node material which extends beyond the bound of the base strut defined by the phantom line 199. For purposes of discussion herein, the inner node material extending between the phantom line 199 and the curved inner surface 198 may be referred to as an intrados region 197.

FIG. 12 illustrates that, for reference purposes, the phantom line 199 may be defined by an inner radius R₃. The inner radius R₃ may be a uniform (e.g., constant) radius whereby the distance from the center point 153 to any point on the phantom line 199 is the same along the curved portion of the phantom line 199. Further, FIG. 12 illustrates that the curved inner surface 198 of the inner node region 196 may include a variable inner radius R₄ whereby the distance from the center point 153 to any point on the curved inner surface 198 changes along the curved inner surface 198. It can be appreciated that both the additional material defining the intrados region 197 in combination with the variable radius configuration of the inner node region 196 may may increase the radial expansion forces of the expandable framework 112 while reducing the structural fatigue of the struts (e.g., struts 168, 164) that are joined together at a given nodal region (e.g., node 192).

FIG. 6 further illustrates the plurality of interconnected strut members forming the distal region 113. It can be appreciated that the distal hub 116 (shown in FIG. 1) may be formed as a separate component during manufacturing of the occlusive implant 100 (shown in FIG. 1), whereby a row of distal hub connecting struts 144 of the distal region 113 are attached to the distal hub 116 in a subsequent manufacturing step after the unitary tubular member is expanded to form the expandable framework 112. In other words, the unitary tubular member may initially be expanded to form the framework 112, after which each of the distal hub connecting struts 144 may be attached to the distal hub 116. Accordingly, for reference purposes, FIG. 6 illustrates the distal hub 116 (shown in FIG. 1) as a phantom line, representing that the row of distal hub connecting struts 144 may be attached to and extend away from the distal hub 116.

FIG. 6 further illustrates that the distal region 113 may include a row of distal engagement nodes 151 separating the engagement region 117 from the distal region 113. Further, FIG. 6 illustrates that the distal region 113 may include a plurality of rows (e.g., a plurality of circumferential rows) of struts extending between the row of distal engagement nodes 151 and the distal hub 116. For example, FIG. 6 illustrates the row of distal hub connecting struts 144 extending from the distal hub 116 to a row of first connecting nodes 143. Further, FIG. 6 illustrates that the distal region 113 may further include a first row of struts 148, a second row of struts 154, a third row of struts 158 and a fourth row of struts 162 positioned between the row of first connecting nodes 143 and the row of distal engagement nodes 151.

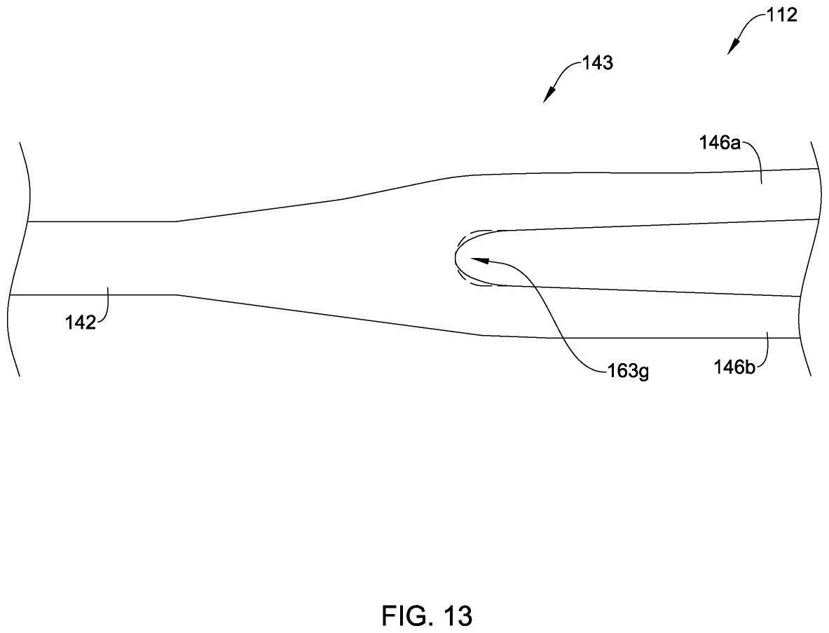

Further, FIG. 6 and FIG. 13 illustrate that a strut 142 of the row of distal hub connecting struts 144 may be connected to a first strut 146a and a second strut 146b of the first row of struts 148 at a first connecting node 143. It can be appreciated that the geometry of the node 143 may include an inner node region 163g which may be similar in form and function to the inner node region 163a described herein. It can be further appreciated that the ratios of the geometric relationships describing the intrados region of node 123 may also apply to the node 143.

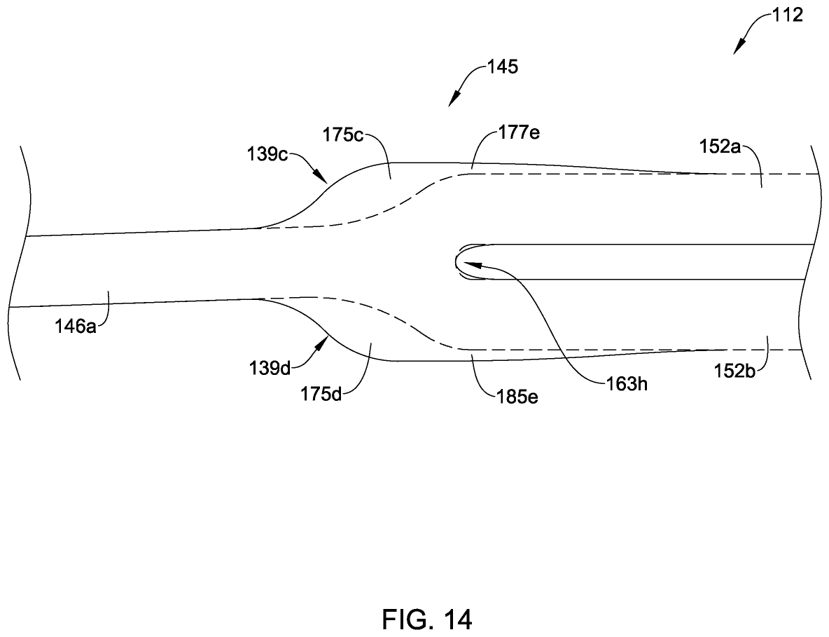

Further, FIG. 6 and FIG. 14 illustrate that a first strut 146a of the first row of struts 148 may be connected to a first strut 152a and a second strut 152b of the second row of struts 154 at a second connecting node 145. It can be appreciated that the first strut 146a may be slightly offset or angled with respect to the longitudinal axis of the first strut 152a and a second strut 152b. Further, for clarity purposes, the second strut 146b is not shown in FIG. 14, however, it can be appreciated that the second strut 146b of the first row of struts 148 may also be connected to a first strut 152a and a second strut 152b of the second row of struts 154 at a second connecting node 145 in a similar configuration as shown with respect to FIG. 13.

Additionally, it can be appreciated that the geometry of the node 145 may include extrados material 177e, 185e and an inner node region 163h which may be similar in form and function to the extrados region 177a and the inner node region 163a described herein. It can be further appreciated that the ratios of the geometric relationships describing the extrados and intrados regions of node 123 may also apply to the node 145. Further, FIG. 14 illustrates that the node 145 may include transition extrados lines 139c, 139d which may include a similar but slightly modified shapes when compared to the transition extrados line 139a described herein. For example, the transition extrados lines 139c, 139d may be configured such that they defines relatively greater transition volumes of extrados material 175c, 175d compared to the transition volume of extrados material 175a described herein.

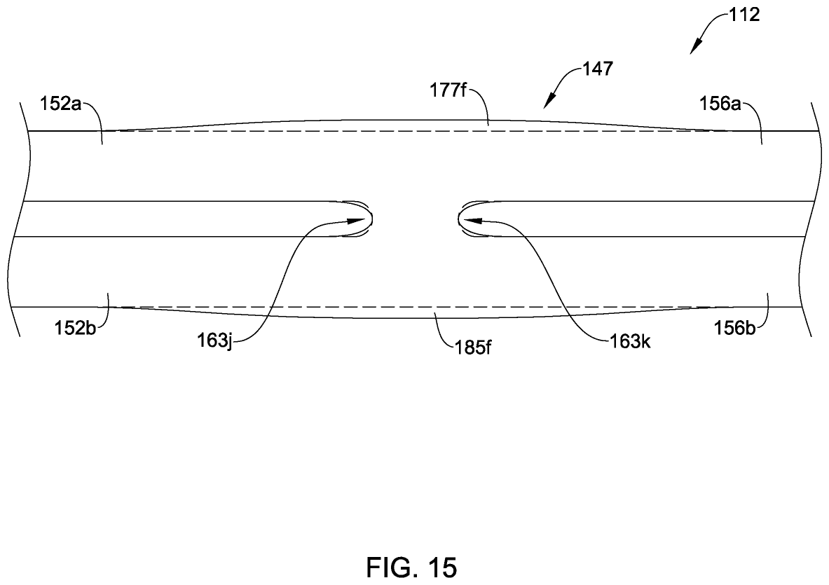

Further, FIG. 6 and FIG. 15 illustrate that the first strut 152a and the second strut 152b of the second row of struts 154 may be connected to a first strut 156a and a second strut 156b of the third row of struts 158 at a third connecting node 147. It can be appreciated that the geometry of the node 147 may include extrados material 177f, 185f and inner node regions 163j, 163k which may be similar in form and function to the extrados region 177a and inner node region 163a described herein. It can be further appreciated that the ratios of the geometric relationships describing the extrados and intrados regions of node 123 may also apply to the node 147.

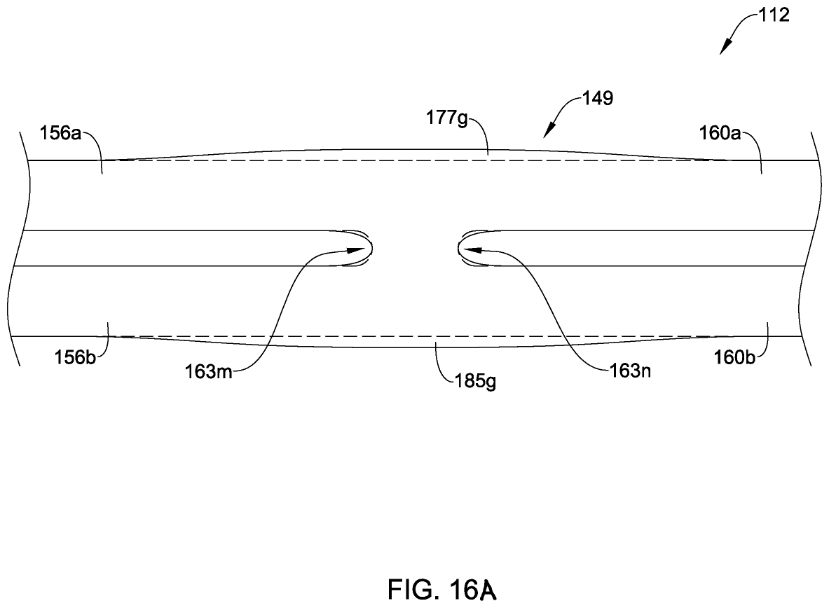

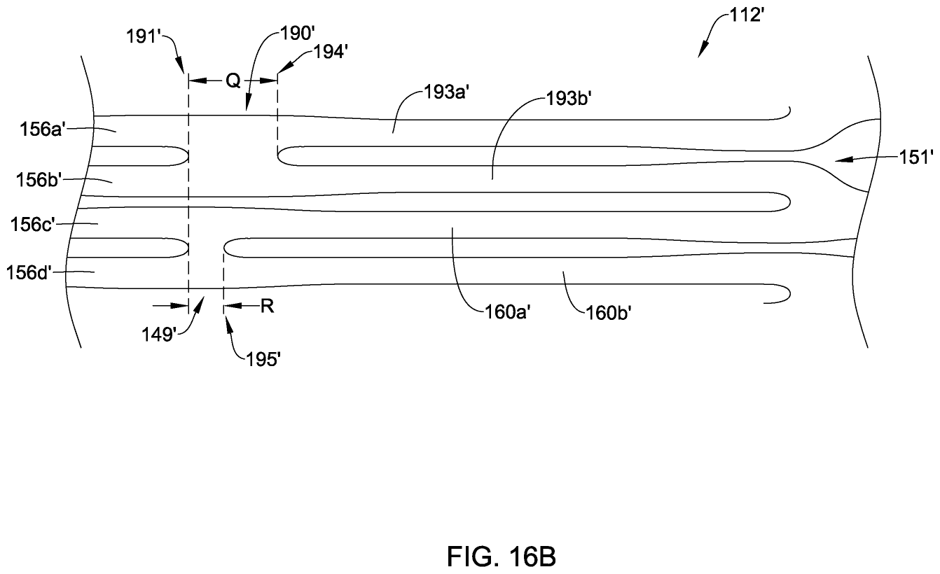

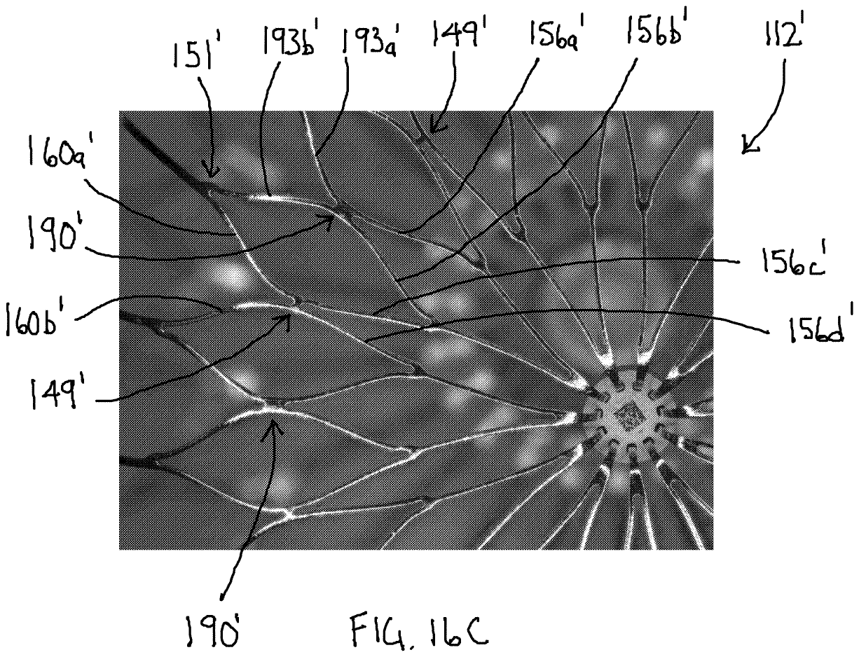

Further, FIG. 6 and FIG. 16A illustrate that the first strut 156a and the second strut 156b of the third row of struts 158 may be connected to a first strut 160a and a second strut 160b of the fourth row of struts 162 at a fourth connecting node 149. It can be appreciated that the geometry of the node 149 may include extrados material 177g, 185g and inner node regions 163m, 163n which may be similar in form and function to the extrados region 177a and inner node region 163a described herein. It can be further appreciated that the ratios of the geometric relationships describing the extrados and intrados regions of node 123 may also apply to the node 149.

FIGS. 16B-16C illustrate a portion of an example expandable framework 112′ which may be an alternative embodiment of the expandable framework 112 described herein. The expandable framework 112′ may be similar in form and function to the expandable framework 112 described herein. For example, the expandable framework 112′ may include a node 149′. The node 149′ may be similar in form and function to the node 149 described herein. For example, the ratios of the geometric relationships describing the extrados and intrados regions of node 149 may also apply to the node 149′. FIG. 16B further illustrates that the expandable framework 112′ may include a strut 156c′ (similar in form and function to struts 156 described herein) and a strut 156d′ (similar in form and function to struts 156 described herein) may be connected to a strut 160a′ and a strut 160b′ at the connecting node 149′. FIG. 16B further illustrates that the connecting node 149′ may include a node length “R” shown extending from the reference line 191′ to the reference line 195′. In some examples, the node length R may be about 0.005 inches to about 0.250 inches, or about 0.010 inches to about 0.250 inches, or about 0.025 inches to about 0.225 inches, or about 0.050 inches to about 0.200 inches. Further, in some examples, the length of the struts 160a′ and 160b′ may be about 0.005 inches to about 0.250 inches, or about 0.010 inches to about 0.250 inches, or about 0.025 inches to about 0.225 inches, or about 0.050 inches to about 0.200 inches.

Additionally, FIG. 16B illustrates that the expandable framework 112′ may include a strut 156a′ (similar in form and function to struts 156 described herein) and a strut 156b′ (similar in form and function to struts 156 described herein) connected to a strut 193a′ and a strut 193b′ at a connecting node 190′. The node 190′ may be similar in form and function to the node 149 described herein. For example, the ratios of the geometric relationships describing the extrados and intrados regions of node 149 may also apply to the node 190′. However, FIG. 16B further illustrates that the connecting node 190′ may include a node length “Q” shown extending from the reference line 191′ to the reference line 194′. In some examples, the node length Q may be about 0.005 inches to about 0.250 inches, or about 0.010 inches to about 0.250 inches, or about 0.025 inches to about 0.225 inches, or about 0.050 inches to about 0.200 inches. Further, in some examples, the length of the struts 193a′ and 193b′ may be about 0.005 inches to about 0.250 inches, or about 0.010 inches to about 0.250 inches, or about 0.025 inches to about 0.225 inches, or about 0.050 inches to about 0.200 inches.

It can be appreciated from FIG. 16B that the node length Q of the connecting node 190′ may be longer than the node length R of the connecting node 149′ when measured from the reference line 191′. Further, FIG. 16C illustrates that the expandable framework 112′ may be configured to alternate the relatively longer length connecting nodes 190′ with the relatively shorter length connecting nodes 149′ along their respective circumferential row of connecting nodes. This alternating node configuration may extend around the circumference of the expandable framework 112′. It can be appreciated that configuring an expandable framework (e.g., expandable framework 112′) to include an alternating node configuration (e.g., connecting nodes having relatively longer node length alternating with connecting nodes having a relatively shorter node length) along a given row of connecting nodes may promote a more uniform strut and cell pattern upon expansion of the expandable framework 112′ during the manufacturing process. In other words, an alternating node configuration may promote “expansion uniformity” whereby the relative position and arrangement of the struts are uniform across the strut pattern during expansion of the expandable framework 112′.

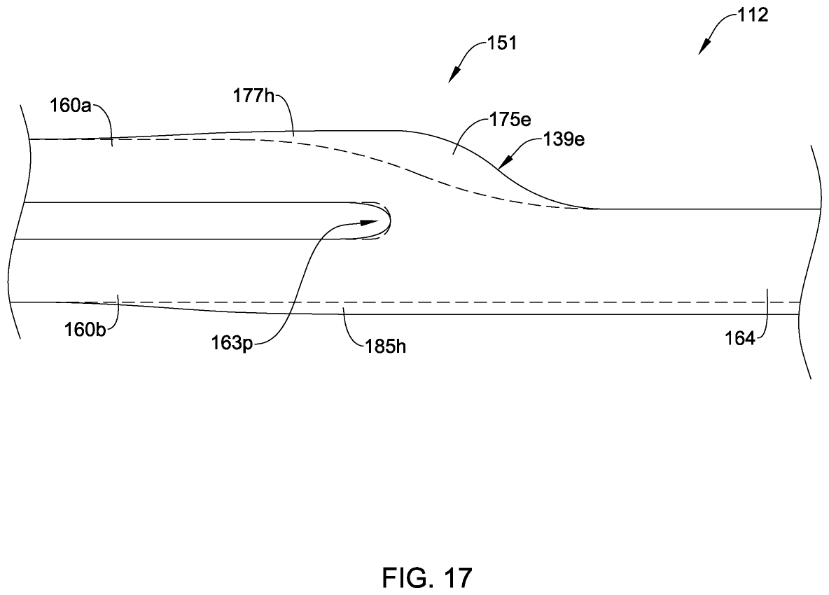

FIG. 6 and FIG. 17 illustrate that the first strut 160a and the second strut 160b of the fourth row of struts 162 may be connected to a strut 164 of the engagement region 117 at a distal engagement node 151. It can be appreciated that the geometry of the node 151 may include extrados material 177h, 185h and inner node region 163p which may be similar in form and function to the extrados region 177a and inner node region 163a described herein. It can be further appreciated that the ratios of the geometric relationships describing the extrados and intrados regions of node 123 may also apply to the node 151. Further, FIG. 11 illustrates that the node 151 may include a transition extrados line 139e which may include a similar but slightly modified shape when compared to the transition extrados line 139a described herein. For example, the transition extrados line 139e may be configured such that it defines a relatively greater transition volume of extrados material 175e compared to the transition volume of extrados material 175a described herein.

It can be appreciated that the number of struts in each row of struts 144, 148, 154, 158, 162 may be the same or may differ from one another. For example, the number of the struts 142 of the row of distal hub connecting struts 144 may include 9 struts to 68 struts, the number of the struts 146 of the first row of struts 148 may include 18 struts to 68 struts, the number of the struts 152 of the second row of struts 154 may include 36 struts to 68 struts, the number of the struts 156 of the third row of struts 158 may include 36 struts to 68 struts and the number of the struts 160 of the fourth row of struts 162 may include 36 struts to 68 struts.

Further, FIG. 6 illustrates that the lengths of the struts 142 of the row of distal hub connecting struts 144 may be shorter than the struts 146 of the first row of struts 148, the struts 152 of the second row of struts 154, the struts 156 of the third row of struts 158 and the struts 160 of the fourth row of struts 162. Further, the lengths of the struts 146 of the first row of struts 148 may be longer than the struts 142 of the row of distal hub connecting struts 144 while being shorter than the struts 152 of the second row of struts 154, the struts 156 of the third row of struts 158 and the struts 160 of the fourth row of struts 162. Further, the struts 152 of the second row of struts 154, the struts 156 of the third row of struts 158 and the struts 160 of the fourth row of struts 162 may all be about the same length as one another while being longer than the struts 146 of the first row of struts 148 and the struts 142 of the row of distal hub connecting struts 144.

In some examples, the length of the struts 142 of the row of distal hub connecting struts 144 may be about 0.030 inches to about 0.250 inches. In some examples, the length of the struts 146 of the first row of struts 148 may be about 0.030 inches to about 0.250 inches. In some examples, the length of the struts 152 of the second row of struts 154 may be about 0.060 inches to about 0.250 inches. In some examples, the length of the struts 156 of the third row of struts 158 may be about 0.060 inches to about 0.250 inches. In some examples, the length of the struts 160 of the fourth row of struts 162 may be about 0.060 inches to about 0.250 inches.

Further, it can be appreciated that while FIG. 6 illustrates that the distal region 113 of the occlusive implant 100 includes four rows of struts 148, 154, 158, 162 positioned between the row of first connecting nodes 143 and the row of distal engagement nodes 151, other variations are contemplated. For example, the occlusive implant 100 may include 1, 2, 3, 4, 5, 6, 7, 8 or more rows of struts positioned between the row of first connecting nodes 143 and the row of distal engagement nodes 151.

Further, it can be appreciated that while FIG. 6 illustrates that the distal region 113 of the occlusive implant 100 includes five rows of struts 144, 148, 154, 158, 162 positioned between the distal hub 116 and the row of distal engagement nodes 151, other variations are contemplated. For example, the occlusive implant 100 may include 1, 2, 3, 4, 5, 6, 7, 8 or more rows of struts positioned between the distal hub 116 and the row of distal engagement nodes 151.

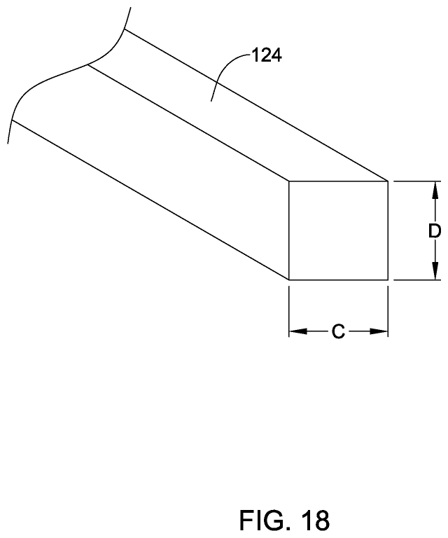

FIG. 18 illustrates a cross-sectional view of a strut 124 of the first row of struts 126 shown in FIG. 6. It can be appreciated from FIG. 18 that the strut 124 may include a width “C” and a thickness “D”. It can be appreciated that the thickness D may correspond to the thickness of the unitary tubular member which is expanded into the expandable framework 112 as described herein. In some examples, the width C of the strut 124 may be about 0.0025 inches to about 0.020 inches. Further, in some examples, the thickness D of the strut 124 may be about 0.004 inches to about 0.010 inches. Accordingly, it can be appreciated that, in some examples, the ratio of the width C to the thickness D of the strut 124 may be about 0.25 to about 5, or about 0.25 to about 2.5, or about 0.35 to about 2.0, or about 0.50 to about 3.0, or about 0.75 to about 1.25, or about 0.60 to about 1.10, or about 0.65 to about 0.75, or about 0.70 to about 1.0. It can be appreciated that the relative dimensions disclosed with respect to the strut 124 in FIG. 18 may also be applied to the other struts of the proximal region 115 and/or the distal region 113. For example, each of the struts 120, 130, 134, 138, 142, 146, 152, 156, 160 may include a width C of about 0.0025 inches to about 0.020 inches and a thickness D of about 0.004 inches to about 0.010 inches. Accordingly, it can be appreciated that the ratio of the width C to the thickness D of the struts 120, 130, 134, 138, 142, 146, 152, 156, 160 may be about 0.25 to about 5, or about 0.25 to about 2.5, or about 0.35 to about 2.0, or about 0.50 to about 3.0, or about 0.75 to about 1.25, or about 0.60 to about 1.10, or about 0.65 to about 0.75, or about 0.70 to about 1.0. Additionally, in some examples, one or more of the struts 120, 124 130, 134, 138, 142, 146, 152, 156, 160 may include a width and/or a thickness which varies from one or more of the struts 120, 124 130, 134, 138, 142, 146, 152, 156, 160. In other words, each strut may 120, 124 130, 134, 138, 142, 146, 152, 156, 160 may have a different width and/or thickness.

The labels of the rows of struts of the distal region 113, the proximal region 115 and/or the engagement region 117 are merely exemplary whereby other structures of the device 100 could bear these names.

FIG. 6 further illustrates the plurality of interconnected strut members forming the engagement region 117. FIG. 6 illustrates the fixation members 166, 168 disposed along one or more struts 164 of the expandable framework 112. It can be appreciated that the number of engagement struts 164 may include 18 to 34 engagement struts. In some examples, the length of the struts 164 of the engagement region 117 may be about 0.100 inches to about 0.600 inches.

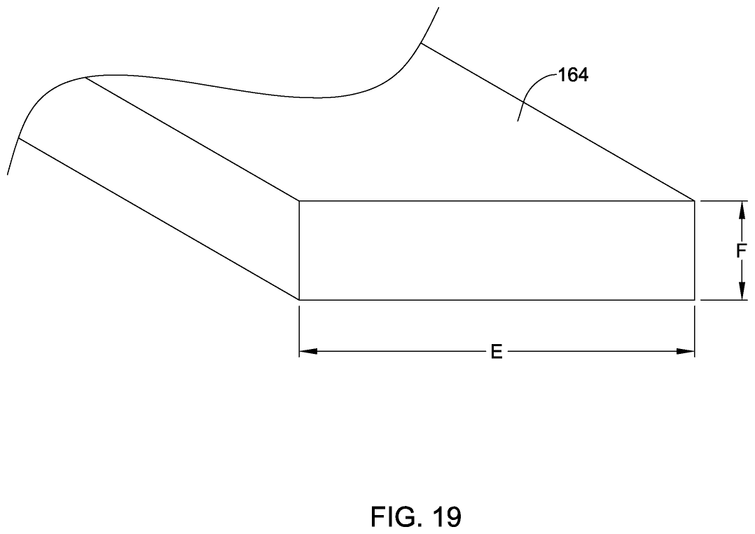

FIG. 19 illustrates a cross-sectional view of a strut 164 shown in FIG. 6. It can be appreciated from FIG. 19 that the strut 164 may include a width “E” and a thickness “F”. In some examples, the width E of the strut 164 may be about 0.0025 inches to about 0.020 inches. Further, in some examples, the thickness F of the strut 164 may be about 0.004 inches to about 0.010 inches. Accordingly, it can be appreciated that, in some examples, the ratio of the width to the thickness of the strut 164 may be about 0.25 to about 5, or about 0.25 to about 2.5, or about 0.35 to about 2.0, or about 0.50 to about 3.0, or about 0.75 to about 1.25, or about 0.60 to about 1.10, or about 0.65 to about 0.75, or about 0.70 to about 1.0.

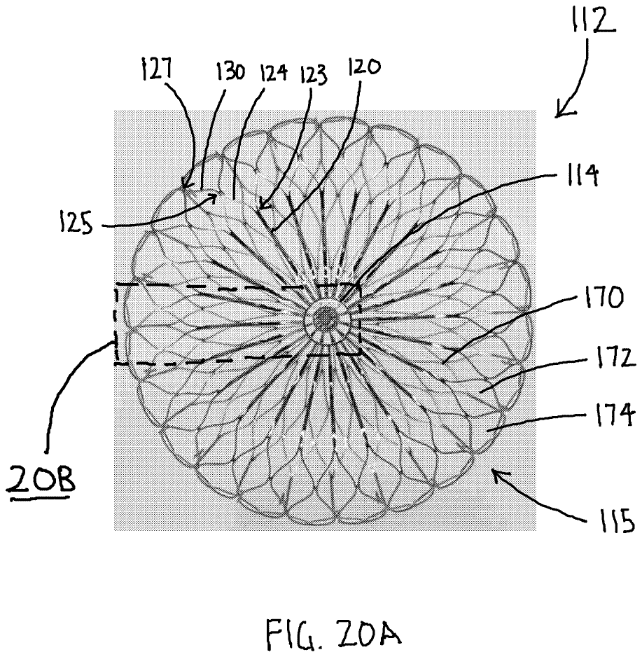

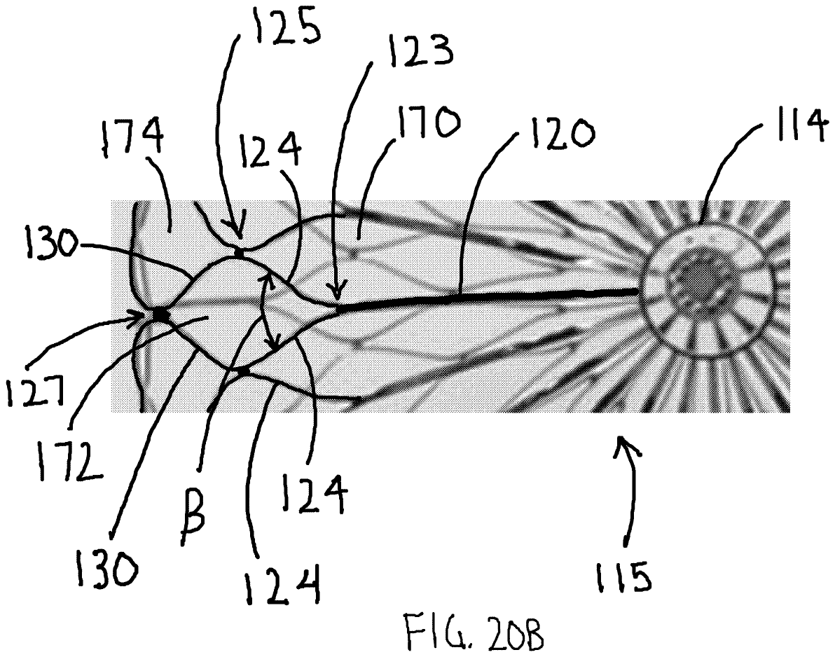

FIG. 20A is a view facing the proximal region 115 of the occlusive implant 100 shown in FIG. 1. FIG. 20A illustrates the plurality of interconnected strut members forming the proximal region 115, whereby the plurality of interconnected strut members extend away from the proximal hub 114. Further, FIG. 20A illustrates that the plurality of interconnected strut members forming the proximal region 115 may extend circumferentially around the longitudinal axis 118 (shown in FIG. 1) of the expandable framework 112.

FIG. 20B illustrates a detailed view of FIG. 20A. FIGS. 20A-20B further illustrate the struts 120 described above with respect to FIG. 1 and FIG. 6. The plurality of struts 120 may each have a first end connected to the proximal hub 114 and a second end which may be connected to a first end of each of two struts 124 at a connecting node 123. Further, each of the struts 124 may have a second end which may be connected to a first end of each of two struts 130 at a connecting node 125. Further, each of the struts 130 may have a second end which may be connected to a first end of each of two struts 134 (shown in FIG. 1) at a connecting node 127. Further, each of the struts 134 may have a second end which may be connected to a first end of each of two struts 138 (shown in FIG. 1) at a connecting node 129. Further, each of the struts 138 may have a second end which may be connected to a first end of strut 164 (shown in FIG. 1) at a proximal engagement node 131.

It can be appreciated that the combination of struts 120 and struts 124 may define a plurality of interstitial cells 170 forming a first row of interstitial cells, the combination of struts 124 with struts 130 may define a plurality of interstitial cells 172 forming a second row of interstitial cells, the combination of struts 130 with struts 134 (shown in FIG. 1) may define a plurality of interstitial cells 174 (shown in FIG. 1) forming a third row of interstitial cells, and combination of struts 134 (shown in FIG. 1) with struts 138 (shown in FIG. 1) may define a plurality of interstitial cells 176 (shown in FIG. 1) forming a fourth row of interstitial cells of the proximal region 115 of the expandable framework 112.

FIG. 1 and FIG. 20A illustrate the expandable framework 112 including struts 120, 124, 130, 134, 138 which form four rows of interstitial cells of the proximal region 115. However, it is contemplated that the expandable framework 112 may include any number of struts to form 1, 2, 3, 4, 5, 6, 7, 8 or more rows of interstitial cells. It can be appreciated that configuring the proximal region 115 to include more (e.g., additional) struts which form relatively more interstitial rows of cells may permit the proximal region 115 of the expandable framework 112 to maintain an expanded, fully deployed configuration (e.g., deployed to its fully expanded diameter) despite the distal region 113 being held in a compressed configuration because the additional strut members have circumferential elements that apply an increased hoop force (e.g., hoop strength) to help expand the proximal region 115 of the expandable frame 112. Increasing the number of strut members in the proximal region 115 may permit the proximal region 115 to articulate, expand and maintain its configuration in localized regions of the expandable frame 112 despite the distal region 113 being held in a compressed configuration.

Additionally, FIG. 20B illustrates an angle β formed between two adjacent struts 124. In some examples, the angle β may be between about 40 and 80 degrees. In some examples, the angle β may be between about 40 and 80 degrees, or between about 45 and 75 degrees, or between about 50 and about 70 degrees, or between about 55 and about 65 degrees. It can be appreciated that increasing the degree of the angle β between any two adjacent strut members 120, 124, 130, 134, 138 forming the proximal region 115 (e.g., increasing the angle β between adjacent struts 124 from about 40 degrees to about 80 degrees) may permit the proximal region 115 to expand in localized regions of the expandable frame 112 despite the distal region 113 being held in a compressed (e.g., static, pinched) state. Additionally, it can be appreciated that angle β between adjacent struts may allow the proximal region 115 to include an increased number of total struts in the proximal region 115. As discussed, increasing the total number of struts forming the proximal region 115 may permit the proximal region 115 of the expandable framework 112 to exert a greater hoop force, thereby allowing the proximal region 115 to maintain an expanded, fully deployed configuration (e.g., deployed to its fully expanded diameter) despite the distal region 113 being held in a compressed configuration.

Further, it can be appreciated that, in some examples, decreasing the wall thickness of one or more of the plurality of struts forming the distal region 113 and/or the proximal region 115 may also provide a more flexible strut geometry which may permit the distal region 113 to expand despite the proximal region 115 being held in a compressed configuration and/or may permit the proximal region 115 to expand despite the distal region 113 being held in a compressed configuration. Configuring the plurality of struts forming the proximal region 115 to have a wall thickness which is approximately 0.004 to about 0.010 inches may increase the flexibility and compliance of the expandable framework 112 of the occlusive implant 100.

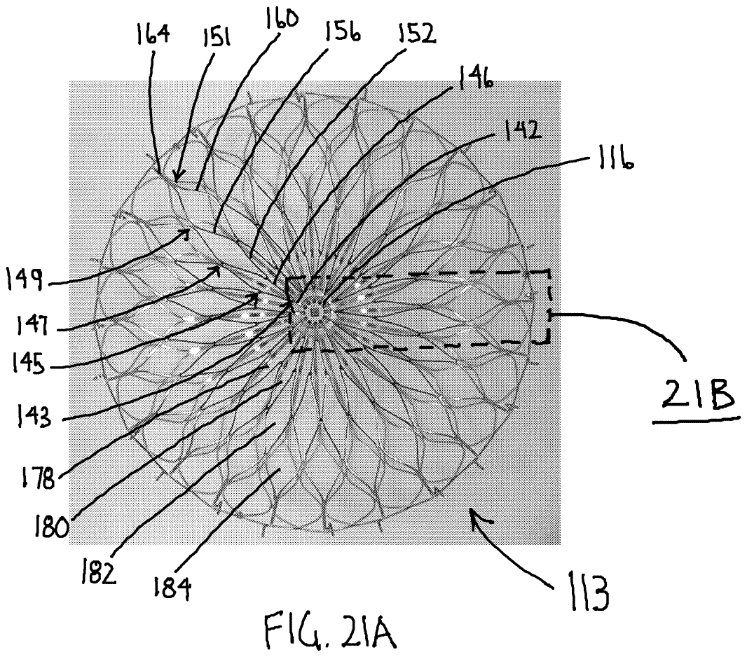

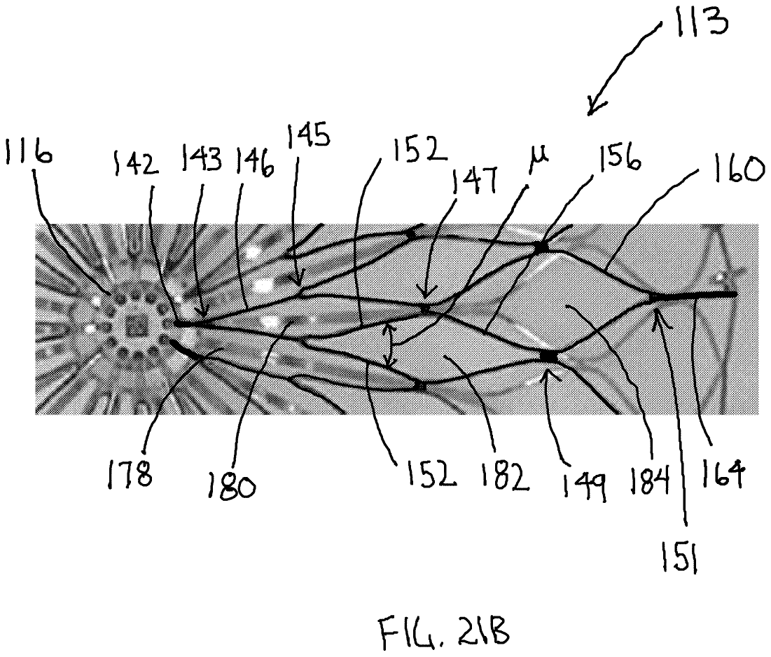

FIG. 21A is a view facing the distal region 113 of the occlusive implant 100 shown in FIG. 1. FIG. 21A illustrates the plurality of interconnected strut members forming the distal region 113, whereby the plurality of interconnected strut members extend away from the distal hub 116. Further, FIG. 21A illustrates that the plurality of interconnected strut members forming the distal region 113 may extend circumferentially around the longitudinal axis 118 of the expandable framework 112.

FIG. 21B illustrates a detailed view of FIG. 21A. FIGS. 21A-21B illustrate struts 142 may each have a first end connected to the distal hub 116 and a second end which may be connected to a first end of each of two struts 142 at a connecting node 143. Further, each of the struts 146 may have a second end which may be connected to a first end of each of two struts 152 at a connecting node 145. Further, each of the struts 152 may have a second end which may be connected to a first end of each of two struts 156 at a connecting node 147. Further, each of the struts 156 may have a second end which may be connected to a first end of each of two struts 160 at a connecting node 149. Further, each of the struts 160 may have a second end which may be connected to a first end of strut 164 (also shown in FIG. 1) at a distal engagement node 151.

It can be appreciated that the combination of struts 142, with struts 146 and struts 152 may define a plurality of interstitial cells 178 forming a first row of interstitial cells, the combination of struts 146 with struts 152 may define a plurality of interstitial cells 180 forming a second row of interstitial cells, the combination of struts 152 with struts 156 may define a plurality of interstitial cells 182 forming a third row of interstitial cells, and the combination of struts 156 with struts 160 may define a plurality of interstitial cells 184 forming a fourth row of interstitial cells in the distal region 113 of the expandable framework 112.

FIGS. 21A-21B illustrates the expandable framework 12 including struts 142, 146, 152, 156, 160 which form four rows of interstitial cells in the distal region 113 of the expandable framework 112. However, it is contemplated that the distal region 113 of the expandable framework 112 may include any number of struts to form 1, 2, 3, 4, 5, 6, 7, 8 or more rows of interstitial cells. It can be appreciated that configuring the distal region 113 to include more (e.g., additional) struts which form relatively more interstitial rows of cells may permit the distal region 113 of the expandable framework 112 to maintain an expanded, fully deployed configuration (e.g., deployed to its fully expanded diameter) despite the distal region 113 being held in a compressed configuration because the additional strut members have circumferential elements that apply an increased hoop force (e.g., hoop strength) to help expand the distal region 113 of the expandable frame 112. Increasing the number of strut members in the distal region 113 may permit the distal region 113 to articulate, expand and maintain its configuration in localized regions of the expandable frame 112 despite the proximal region 115 being held in a compressed configuration.

Additionally, FIG. 21B illustrates that angle μ being formed between two adjacent struts 152. In some examples, the angle μ may be between about 40 and 80 degrees, or between about 45 and 75 degrees, or between about 50 and about 70 degrees, or between about 55 and about 65 degrees. It can be appreciated that increasing the degree of the angle μ between any two adjacent strut members 142, 146, 152, 156, 160 forming the distal region 113 (e.g., increasing the angle μ between adjacent struts 152 from about 40 degrees to about 80 degrees) may permit the distal region 113 to expand in localized regions of the expandable frame 112 despite the proximal region 115 being held in a compressed (e.g., static, pinched) state. Additionally, it can be appreciated that angle μ between adjacent struts may allow the distal region 113 to include an increased number of total strut members in the distal region 113. As discussed, increasing the total number of struts forming the distal region 113 may permit the distal region 113 of the expandable framework 112 to exert a greater hoop force, thereby allowing the distal region 113 of the expandable framework 112 to maintain an expanded, fully deployed configuration (e.g., deployed to its fully expanded diameter) despite the distal region 113 being held in a compressed configuration.

Further, it can be appreciated that, in some examples, decreasing the wall thickness of one or more of the plurality of struts forming the distal region 113 and/or the proximal region 115 may also provide a more flexible strut geometry which may permit the distal region 113 to expand despite the proximal region 115 being held in a compressed configuration and/or may permit the proximal region 115 to expand despite the distal region 113 being held in a compressed configuration. Configuring the plurality of struts forming the distal region 113 to have a wall thickness which is approximately 0.004 inches to about 0.010 inches may increase the flexibility and compliance of the expandable framework 112 of the occlusive implant 100.

FIGS. 22-23 illustrate a “silhouette” of the expandable framework 112 of the occlusive implant 100 (shown in FIG. 1) in an expanded configuration and including the distal region 113, the proximal region 115 and the engagement region 117. FIG. 22 illustrates that, in some examples, one or more of fixation members 186a (similar in form and function as the fixation members 166, 168 described herein) may be positioned along the distal region 113 of the expandable framework 112 and fixation members 186b (similar in form and function as the fixation members 166, 168 described herein) may be positioned along the proximal region 115 of the expandable framework 112. FIG. 22 illustrates that, when positioned along the proximal region 115, the fixation members 186b may point toward the distal region 113. For example, as illustrated in FIG. 22, the fixation members 186b may point toward the fixation members 186a and the fixation members 186a may point toward the fixation members 186b. It can be appreciated that the fixation members 186b may be positioned along any portion of the engagement region 117 and/or proximal region 115 of the expandable framework 112. It can be further appreciated that the fixation members 186b may be arranged in one or more horizontal rows along the engagement region 117 and/or the proximal region 115. It can be appreciated that the fixation members 186b may be vertically offset from one another along the engagement region 117 and/or proximal region 115. Additionally, one or more of the fixation members 186b may extend through an occlusive member positioned on the occlusive implant 100.

Additionally, FIG. 23 illustrates that, in some examples, when positioned along the engagement region 117 and/or proximal region 115, the fixation members 186b may point away from the distal region 113. For example, as illustrated in FIG. 23, the fixation members 186b may point in a proximal direction and away from the fixation members 186a, while the fixation members 186a may point in a distal direction and away from the fixation members 186b. It can be appreciated that the configuration shown in FIG. 23 (whereby the fixation members 186a positioned on the distal region 113 point away from the fixation members 186b positioned on the engagement region 117 and/or proximal region 115), may prevent the occlusive implant 100 from sliding into the left atrial appendage in which the occlusive implant 100 is deployed, while also preventing the occlusive implant 100 from sliding out of the left atrial appendage in which the occlusive implant 100 is deployed. Further, it can be appreciated that the fixation members 186b may be positioned along any portion of the engagement region 117 and/or proximal region 115 of the expandable framework 112. It can be appreciated that the fixation members 186b may be arranged in one or more horizontal rows along the engagement region 117 and/or proximal region 115. Additionally, the fixation members 186b may be vertically offset from one another along the engagement region 117 and/or proximal region 115. Further, one or more of the fixation members 186b may extend through an occlusive member positioned on the occlusive implant 100.

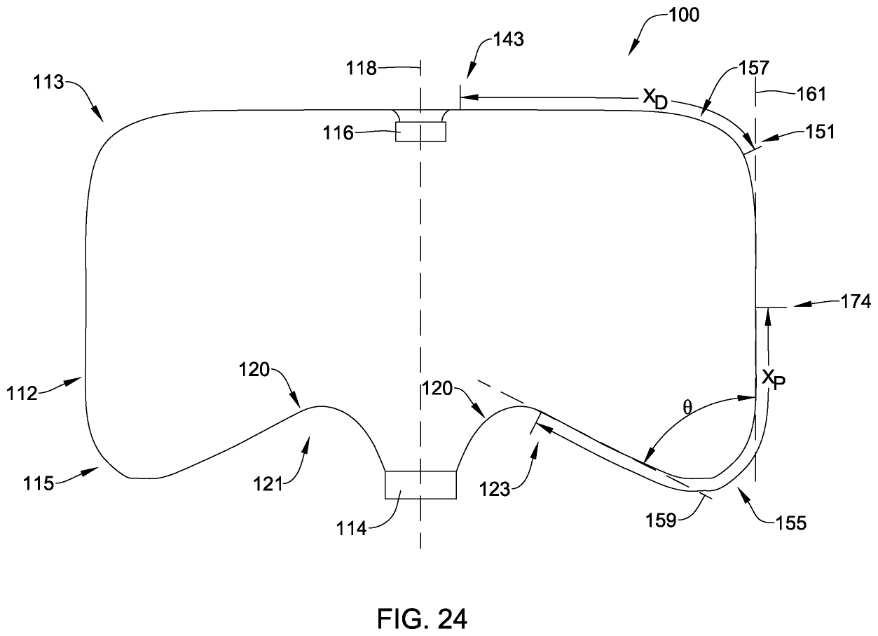

FIG. 24 illustrates a silhouette of the occlusive implant 100 (shown in FIG. 1) in an expanded configuration, including the distal region 113, the proximal region 115, a proximal shoulder 155 and a distal shoulder 157 of the expandable framework 12.

FIG. 24 illustrates two of the plurality of struts 120 of the expandable framework 112 described above with respect to FIG. 1. As discussed herein, the struts 120, 124, 130, 134, 138 (shown in FIG. 1 and FIGS. 20A-20B), may together form the proximal region 115 of the expandable framework 112. Additionally, FIG. 24 illustrates the first ends of each of the struts 120 attached to the proximal hub 114. As discussed herein, the proximal hub 114 may be aligned with the longitudinal axis 118 of the occlusive implant 100 and partially extend into the recess 121 defined by curved portions of the struts 120.

Additionally, FIG. 24 illustrates that a portion of the expandable framework 112 may define a length referred to herein as a “proximal path length” having a length X_{P}. In some instances, the proximal path length X_{P} may extend from the row of first connecting nodes 123 (shown in FIG. 6 and FIGS. 20A-20B) to the row of proximal engagement nodes 131 (shown in FIG. 1 and FIG. 6). Rather than being a path that follows along one or more of the struts defining the proximal region 115, the proximal path length X_{P} may be understood to extend along an imaginary outer surface defined by the framework 112, whereby the proximal path length X_{P} extends in a line that extends along the imaginary surface between the row of first connecting nodes 123 to the row of proximal engagement nodes 131. In some examples, the length of the proximal path length X_{P} may be about 0.40 inches to 1.6 inches. It can be appreciated from FIG. 6 that the first row of struts 126, the second row of struts 132, the third row of struts 136 and the fourth row of struts 138 of the proximal region 115 may lie within the proximal path length X_{P}.

FIG. 24 further illustrates the struts 142, 146, 152, 156, 160 (shown in FIGS. 21A-21B) may together form the distal region 113 of the expandable framework 112. Additionally, FIGS. 21A-21B illustrate the first ends of each of the struts 142 attached to the distal hub 116. The distal hub 116 may be aligned with the longitudinal axis 118 of the occlusive implant 100. Additionally, FIG. 24 illustrates that a portion of the expandable framework 112 may define a length referred to herein as a “distal path length” having a length X_{D}. In some instances, the distal path length X_{D} may extend from the distal hub 116 to the row of distal engagement nodes 151 (shown in FIG. 6 and FIGS. 21A-21B). Rather than being a path that follows along one or more of the struts defining the distal region 113, the distal path length X_{D} may be understood to extend an imaginary outer surface defined by the framework 112, whereby the distal path length X_{D} extends in a line that extends along the imaginary surface between the distal hub 116 to the row of distal engagement nodes 151 (shown in FIG. 6 and FIGS. 21A-21B). In some examples, the length of the distal path length X_{D} may be about 0.10 inches to 0.80 inches. It can be appreciated from FIG. 6 that the row of distal hub connecting struts 144, first row of struts 148, the second row of struts 154, the third row of struts 158 and the fourth row of struts 162 of the distal region 113 may lie within the distal path length X_{D}.

Further, FIG. 24 illustrates that the strut 120 lies along an axis illustrated by the dashed line 159. It can be appreciated that an axis 161 and the axis 159 may define an interior shoulder angle θ extending therebetween. In some examples, the shoulder angle θ of the expandable framework 112 shown in FIG. 24 may be about 45 to about 55 degrees, or about 48 to 52 degrees, or about 50 degrees.

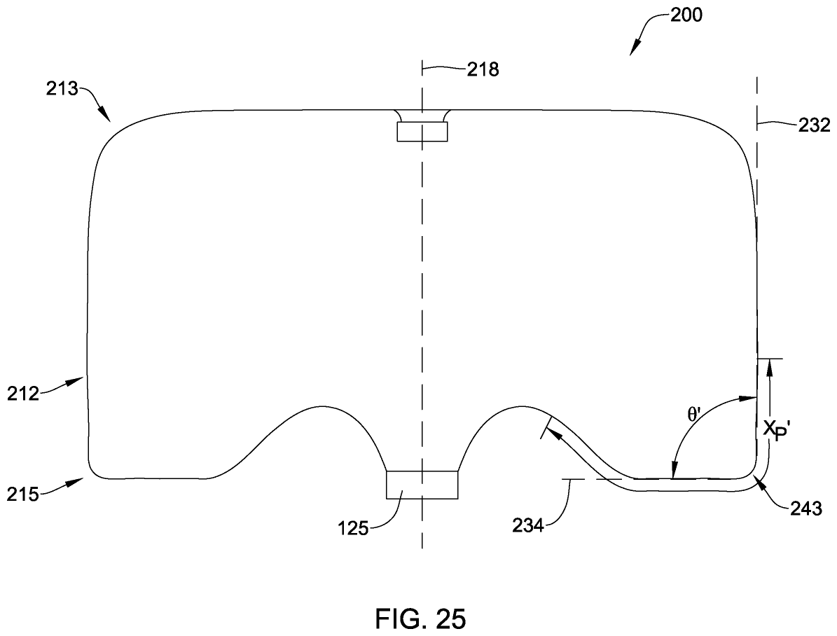

FIG. 25 illustrates another example expandable occlusive implant 200 which may be similar in form and function to the occlusive implant 100 described herein. For example, the occlusive implant 200 may include a framework 212 having a plurality of strut members (e.g., struts) forming a distal region 213. The expandable framework may also include a plurality of strut members forming a proximal region 215. The strut members forming the distal region 213 may be interconnected with one another and extend circumferentially around the central longitudinal axis 218 to define the distal region 213 of the expandable framework 212 of the occlusive implant 200.

The occlusive implant 200 may include features which may permit the proximal region 215 of an expandable framework 212 to maintain an expanded, fully deployed configuration (e.g., deployed to its fully expanded diameter) despite the distal region 213 being held in a compressed configuration. FIG. 25 illustrates an axis illustrated by the dashed line 232 and an axis illustrated by the dashed line 234, whereby the axis 232 and the axis 234 may define an interior shoulder angle θ′ extending therebetween. For example, compared to the occlusive implant 100 illustrated in FIG. 24, the occlusive implant 200 of FIG. 25 includes an increased (e.g., larger) shoulder angle θ′ compared to the shoulder angle θ shown in FIG. 24. Additionally, it can be appreciated that the shoulder 243 may further include a tighter radius compared to the shoulder 155 shown in FIG. 24. In some examples, the shoulder angle θ′ of the expandable framework 212 shown in FIG. 25 may be about 85 to about 95 degrees, or about 88 to 92 degrees, or about 90 degrees.

It can be appreciated that configuring the proximal region 215 of the expandable framework 212 to include a relatively larger shoulder angle θ′ (e.g., about 50-90 degrees) and tighter radius may permit the proximal region 215 to preserve its expanded shape despite the distal region 213 being pinched, constrained or held in a fixed position. For example, configuring the shoulder 243 to include a larger shoulder angle θ′ and/or a tighter radius may permit the proximal region 215 to expand and flare outward when the distal region 213 is constrained within a portion of the left atrial appendage.

Additionally, FIG. 25 illustrates that increasing the shoulder angle of shoulder 243 may also increase the proximal path length X_{P} (shown in FIG. 24) to the path length X_{P}′ (shown in FIG. 25). It can be appreciated that configuring the distal region 213 to include a relatively longer path length X_{P}′ (compared to the path length X_{P} shown in FIG. 24) may permit the proximal region 215 of the expandable member to maintain an expanded, fully deployed configuration (e.g., deployed to its fully expanded diameter) despite the distal region 213 being held in a compressed configuration because the longer path length X_{P}′ enables the expandable framework 212 to elongate and radially expand a greater extent for a given strut length as compared to a relatively shorter path length.

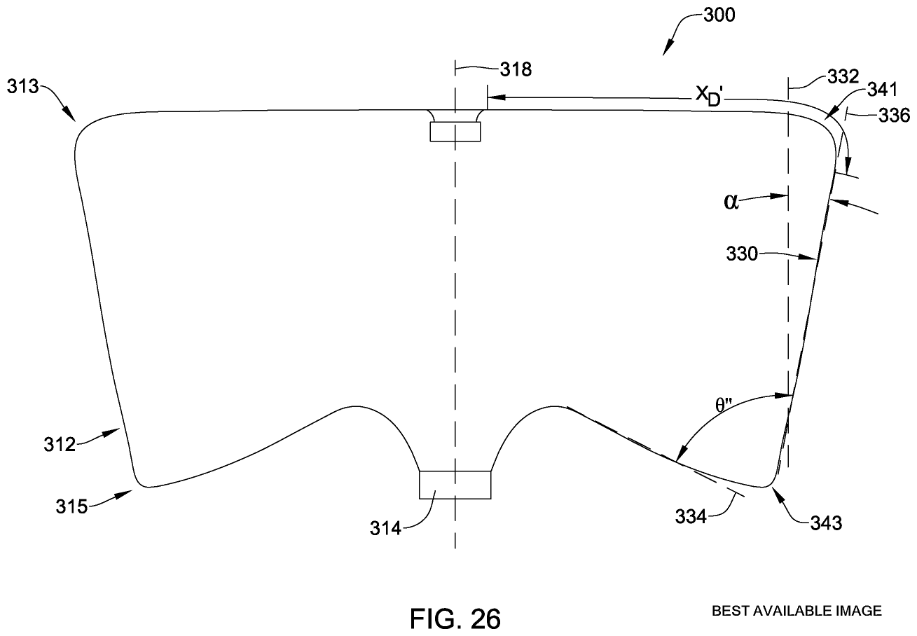

FIG. 26 illustrates another example expandable occlusive implant 300 which may be similar in form and function to the occlusive implant 100 described herein. For example, the occlusive implant 300 may include a framework 312 which includes a plurality of strut members (e.g., struts) forming a distal region 313. The expandable framework 312 may also include a plurality of strut members forming a proximal region 315. The strut members forming the distal region 313 may be interconnected with one another and extend circumferentially around the central longitudinal axis 318 to define the distal region 313 of the expandable framework 312 of the occlusive implant 300.

The occlusive implant 300 may also include features which may permit the distal region 313 of an expandable framework 312 to maintain an expanded, fully deployed configuration (e.g., deployed to its fully expanded diameter) despite the proximal region 315 being held in a compressed configuration. For example, FIG. 26 illustrates that the distal shoulder 341 may include a tighter radius compared to the distal shoulder 157 shown in the expandable framework of FIG. 24. It can be appreciated that configuring the shoulder 341 of the distal region 313 to include a tighter radius may permit the distal region 313 to preserve its expanded shape despite the proximal region 315 being held in a compressed configuration. For example, a tighter radius may permit the shoulder 341 to expand and flare outward when the proximal region 315 is constrained within a portion of the left atrial appendage.

FIG. 26 further illustrates the strut 330 (similar to the strut 164 shown in FIG. 1) of the expandable framework 312 aligned along an axis defined by the dashed line 336. It can be appreciated from comparing the expandable framework 112 shown in FIG. 24 to the expandable framework 312 shown in FIG. 26 that the axis 336 of the strut 330 is offset from the axis 332 (which is parallel to the longitudinal axis 318) by an angle α. In other words, FIG. 26 illustrates an example expandable framework 312 configuration in which the strut 330 has been angled radially away from the longitudinal axis 332 of the expandable framework 312 by an angle α. In some examples, the angle α may be about −4 degrees to about 25 degrees, or about −4 degrees to about 20 degrees, or about 0 degrees to about 15 degrees, or about 0 degrees to about 10 degrees, or about 16 degrees. In some examples, the angle α may be about 0 degrees whereby the strut 330 may substantially align or be substantially parallel with the longitudinal axis 318 and therefor define a “zero taper” configuration which may enhance the ability of the fixation members 166, 168, 186a, 186b to engage, appose and seal against the lateral walls of a left atrial appendage.

Additionally, FIG. 26 illustrates that angling the strut 330 radially away from the longitudinal axis 332 of the expandable framework 312 may also increase the distal path length X_{D} (shown in FIG. 24) to a path length X_{D}′ (shown in FIG. 26). It can be appreciated that the configuration of the framework 312 shown in FIG. 26 (whereby the axis 336 has been angled radially away from the longitudinal axis 332) may permit the path length X_{D} (shown in FIG. 24) to be lengthened to the path length X_{D}′ shown in the configuration of the framework 312 shown in FIG. 26. It can be appreciated that configuring the distal region 313 to include a relatively longer path length X_{D}′ (compared to the path length X_{D} shown in FIG. 24) may permit the distal region 313 of the expandable member to maintain an expanded, fully deployed configuration (e.g., deployed to its fully expanded diameter) despite the proximal region 315 being held in a compressed configuration because the additional path length X_{D}′ enables the expandable framework 312 to elongate and radially expand a greater extent for a given strut length as compared to a shorter path length.

Additionally, it can be appreciated that configuring the distal region 313 to include a relatively longer path length X_{D}′ (compared to the path length X_{D} shown in FIG. 24) may permit the proximal region 315 of the expandable member to maintain an expanded, fully deployed configuration (e.g., deployed to its fully expanded diameter) despite the distal region 313 being held in a compressed configuration because the additional path length X_{D}′ may enable the expandable framework 312 to both elongate and radially expand a greater extent for a given strut length as compared to a shorter path length.

FIG. 26 further illustrates an axis illustrated by the dashed line 334, whereby the axis 336 and the axis 334 may define an interior shoulder angle θ″ extending therebetween. Additionally, it can be appreciated that the shoulder 343 may further include a tighter radius compared to the shoulder 155 shown in FIG. 24. In some examples, the shoulder angle θ″ of the expandable framework 312 shown in FIG. 26 may be about 75 degrees to about 100 degrees, or about 80 degrees to 95 degrees or about 88 degrees to 92 degrees, or about 90 degrees.

It can be appreciated that configuring the proximal region 315 of the expandable framework 312 to include a larger shoulder angle θ″ (e.g., about 50-90 degrees) and tighter radius may permit the proximal region 315 to preserve its expanded shape despite the distal region 313 being pinched, constrained or held in a fixed position. For example, configuring the shoulder 343 to include a larger shoulder angle θ″ and/or a tighter radius a may permit the proximal region 315 to expand and flare outward when the distal region 313 is constrained within a portion of the left atrial appendage.

Referring to FIG. 24 and FIG. 26, it can be appreciated that for any given occlusive implant (e.g., occlusive implants 100, 200, 300), combining the proximal path length and the distal path length may provide an overall framework path length which, in general, may correspond to the overall length of given occlusive device, whereby a longer overall path length of a device may enable the proximal region and distal region of the expandable framework of the occlusive device to both elongate and radially expand a greater extent for a given strut length as compared to a shorter overall device path length.

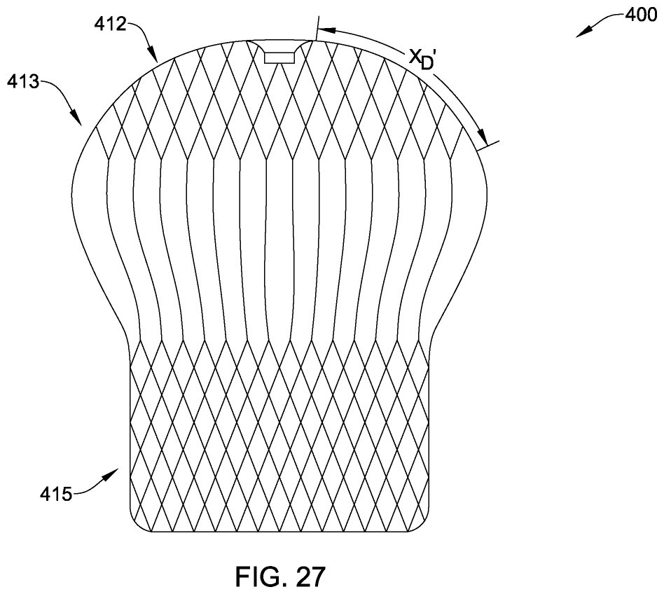

FIG. 27 illustrates another example expandable occlusive implant 400 which may be similar in form and function to the occlusive implant 100 described herein. For example, the occlusive implant 400 may include a framework 412 which includes features which may permit a distal region 413 of an expandable framework 412 to maintain an expanded, fully deployed configuration (e.g., deployed to its fully expanded diameter) despite a proximal region 415 being held in a compressed configuration. As discussed herein, it can be appreciated that increasing the path length X_{D}′ of the struts which form a portion of the distal region 413 may allow for increased distal expansion of the distal region 413 of the expandable framework 412. The increased distal expansion may provide a more flexible strut geometry of the distal region 413 relative to the proximal region 415 of the expandable framework 412, as discussed herein.

FIG. 27 further illustrates that the distal region 413 of the occlusive implant 400 may expand radially outward such that it forms a substantially round shape. It can be appreciated that the flexibility and compliance of the strut geometry of the distal region 413 of the expandable framework 412 may permit the distal region 413 to expand into a relatively round shape, as shown in FIG. 27. The round shape of the distal region 413 may result in the distal region 413 being more conformable to the shape of the walls defining a left atrial appendage within which the expandable frame 412 is deployed.

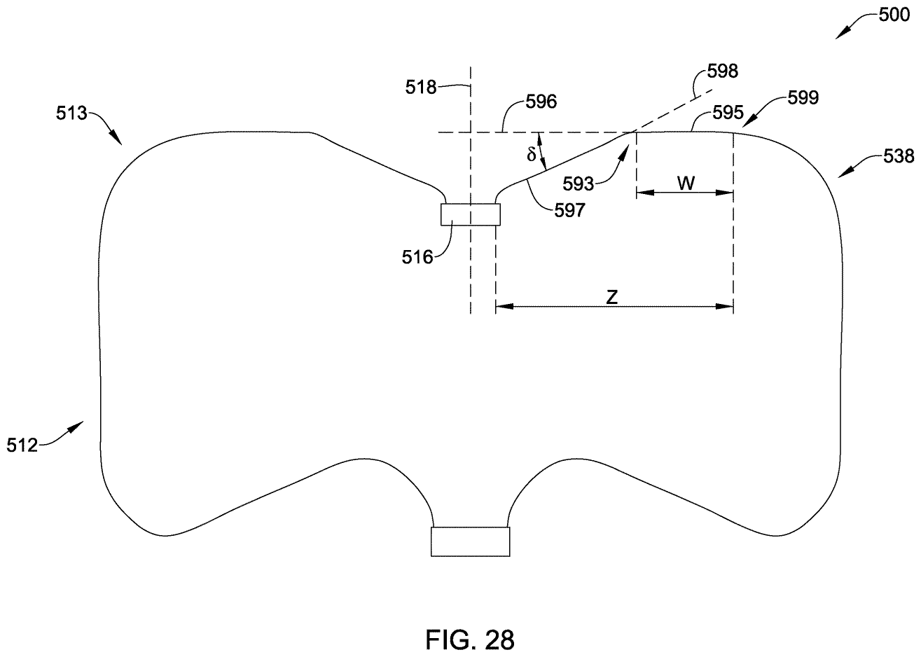

FIG. 28 illustrates another example expandable occlusive implant 500 which may be similar in form and function to the occlusive implant 100 described herein. FIG. 28 illustrates an example configuration of an expandable framework 512 extending around a central longitudinal axis 518, whereby the distal region of the expandable framework 512 may form into a substantially round shape upon expansion (such as the round shape shown in FIG. 27). FIG. 28 illustrates the expandable framework 512 may include a first distal strut member 595 aligned with an axis 596 and a second distal strut member 597 aligned with an axis 598. Additionally, FIG. 28 illustrates that a first end of the strut 597 may be connected to the distal hub 516 and the second end of the strut 597 may be interconnected to a first end of the strut 595. The point 593 where the second end of the strut 597 is connected with the first end of the strut 595 may be referred to herein as a “hinge point” of the distal region 513. FIG. 28 further illustrates a “hinge angle” δ formed between the axis 596 of the strut member 595 and the axis 598 of the strut member 597. In the example illustrated in FIG. 28, the hinge angle δ may be about 5 degrees to about 60 degrees, or between about 10 and about 55 degrees, or between about 15 and about 50 degrees, or between about 20 and about 45 degrees, or between about 25 and about 40 degrees, or between about 30 and about 35 degrees.

Further, a second end of the strut 595 may be defined as the point 599 in which the second end of the strut 595 connects to the distal end of the distal corner portion 538 of the distal region 538. FIG. 28 also illustrates that the expandable framework 512 may also include a first hinge length Z, defined herein as the horizontal distance from the first end of the strut 597 to the second end of the strut 595. FIG. 28 also illustrates that the expandable framework 512 may also include a second hinge length W, defined herein as the horizontal distance from the first end of the strut 595 to the second end of the strut 595. In some examples, configuring the expandable framework 512 to include a hinge angle δ of 5 degrees to about 60 degrees and a ratio of the length W to the length Z

$$( e . g . , Length Ratio = \frac{W}{Z} )$$

of about 0 to 1.0 may permit the distal region 513 to form into a substantially round shape (such as the round shape shown in FIG. 27) upon expansion. The substantially round shape of the distal region 513 may increase the overall flexibility of the distal region 513 of the expandable framework.

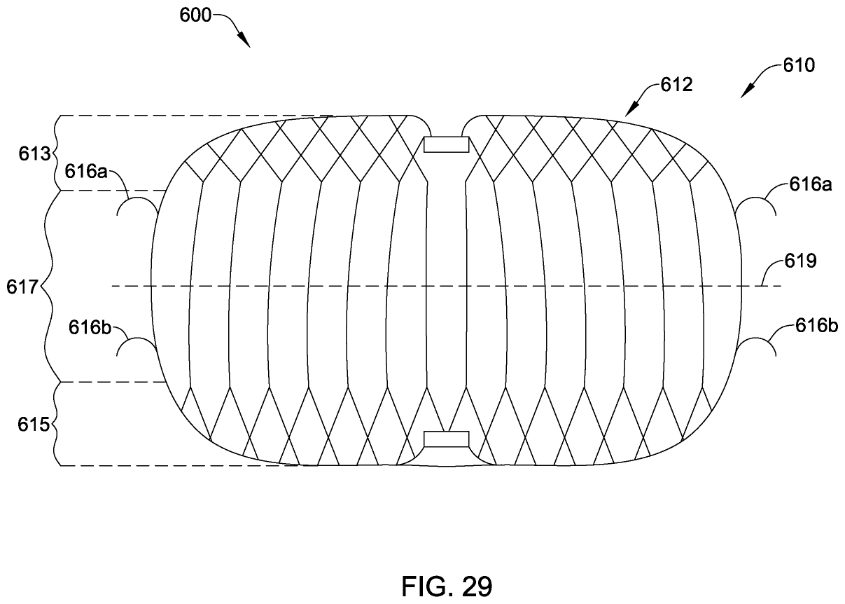

FIG. 29 illustrates another example expandable occlusive implant 600 which may be similar in form and function to the occlusive implant 100 described herein. The occlusive implant 600 may include an expandable framework 612 including a proximal region 615 and a distal region 613. Additionally, the occlusive implant 600 may include an engagement region 617 extending between the proximal region 615 and the distal region 613. In some examples, the engagement region 617 may include a longitudinal extent whereby the mid-point of the longitudinal extent is aligned with the transverse axis 619. In other words, the expandable frame 612 may include a convex mid-body shape, whereby the engagement region 617 curves outward (e.g., bulges outward), away from the longitudinal axis of the occlusive implant 600. FIG. 29 further illustrates that the engagement region 617 may include a diameter which is greater than the diameter of the proximal region 615 and the distal region 613.

FIG. 29 further illustrates that the occlusive implant 600 may include one or more fixation members 616a, 616b (similar in form and function as the fixation members 166, 168, 186a, 186b described herein) positioned along the engagement region 617 expandable framework 612. The plurality of fixation members 616a, 616b disposed along the engagement region 617 of the expandable framework 612 may include several “rows” of fixation members 616a, 616b disposed along the struts forming the engagement region 617. For example, the expandable framework 612 may include several fixation members 616a, 616b disposed along several struts in the engagement region 617. For example, in some instances the expandable framework 612 may include 1, 2, 3, 4, 5, 6, 7, 8, 9, 10, 15, 20, 25, 30, 40, 45, 50 or more fixation members 616a, 616b disposed thereon.

As shown in FIG. 29, each of the fixation members 616a, 616b may be curved such that the tip portion of each fixation member 616a, 616b points toward the proximal region 615 of the expandable framework 612. In other examples, the tips of the fixation members 616a, 616b may point toward one another (e.g., the tip of the fixation member 616a may point toward the tip of the fixation member 616b). In yet other examples, the tips of the fixation members 616a, 616b may point away from one another (e.g., the tip of the fixation member 616a may point away from the tip of the fixation member 616b). Additionally, it is contemplated that the fixation members 616a, 616b may include a variety of different shapes, geometries, etc.

It can be appreciated that positioning the fixation members 616a, 616b along the engagement region 617 may increase the number of fixation member 616a, 616b contact points that the occlusion implant 600 has with the wall of a left atrial appendage within which the occlusive implant 600 may be deployed. In other words, positioning the fixation members 616a, 616b along the outwardly curving portions of the occlusive implant 600 may increase the number of fixation member 616a, 616b contact points that the occlusive implant 600 has with the wall of a left atrial appendage.

The materials that can be used for the occlusive implant 100 (and variations, systems or components thereof disclosed herein) and the various elements thereof disclosed herein may include those commonly associated with medical devices. For simplicity purposes, the following discussion refers to the occlusive implant 100 (and variations, systems or components disclosed herein). However, this is not intended to limit the devices and methods described herein, as the discussion may be applied to other elements, members, components, or devices disclosed herein.

In some embodiments, the occlusive implant 100 (and variations, systems or components thereof disclosed herein) may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (°C) to about 120 °C in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the occlusive implant 100 (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the occlusive implant 100 (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the configuration of the occlusive implant 100 (and variations, systems or components thereof disclosed herein). to achieve the same result.

In some embodiments, a degree of Magnetic Resonance Imaging (MRI) compatibility is imparted into the occlusive implant 100 (and variations, systems or components thereof disclosed herein). For example, the occlusive implant 100 (and variations, systems or components thereof disclosed herein) and/or components or portions thereof, may be made of a material that does not substantially distort the image and create substantial artifacts (e.g., gaps in the image). Certain ferromagnetic materials, for example, may not be suitable because they may create artifacts in an MRI image. The occlusive implant 100 (and variations, systems or components disclosed herein) or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the occlusive implant 100 (and variations, systems or components thereof disclosed herein) and/or portions thereof, may be made from or include a polymer or other suitable material. Some examples of suitable polymers may include copolymers, polyisobutylene-polyurethane, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In some embodiments, the occlusive implant 100 (and variations, systems or components thereof disclosed herein) may include a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun-types. The type and denier of the yarn chosen may be selected in a manner which forms a biocompatible and implantable prosthesis and, more particularly, a vascular structure having desirable properties.

In some embodiments, the occlusive implant 100 (and variations, systems or components thereof disclosed herein) may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

While the discussion above is generally directed toward an occlusive implant for use in the left atrial appendage of the heart, the aforementioned features may also be useful in other types of medical implants where a fabric or membrane is attached to a frame or support structure including, but not limited to, implants for the treatment of aneurysms (e.g., abdominal aortic aneurysms, thoracic aortic aneurysms, etc.), replacement valve implants (e.g., replacement heart valve implants, replacement aortic valve implants, replacement mitral valve implants, replacement vascular valve implants, etc.), and/or other types of occlusive devices (e.g., atrial septal occluders, cerebral aneurysm occluders, peripheral artery occluders, etc.). Other useful applications of the disclosed features are also contemplated.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An occlusive implant, comprising:
a distal hub;
a proximal hub; and
an expandable framework including a plurality of struts spaced around a central longitudinal axis of the occlusive implant and forming a distal region, a proximal region and an engagement region positioned between the distal region and the proximal region;
wherein the plurality of the struts forming the proximal region includes a proximal first row of struts coupled to a row of first connecting nodes, and wherein the proximal first row of struts includes between 18 and 68 struts;
wherein the plurality of the struts forming the distal region includes a distal first row of struts coupled to a row of second connecting nodes, and wherein the distal first row of struts includes between 9 and 68 struts;
wherein the plurality of the struts forming the engagement region includes between 18 and 34 struts;
wherein at least one node of the row of first connecting nodes includes an extrados region, an intrados region or both an extrados region and an intrados region.

2. The occlusive implant of claim 1, wherein at least one node of the row of second connecting nodes includes an extrados region, an intrados region or both an extrados region and an intrados region.

3. The occlusive implant of claim 2, wherein a strut of the plurality engagement struts is coupled to a fixation member at a fixation node, and wherein the fixation node includes an extrados region, an intrados region or both an extrados region and an intrados region.

4. The occlusive implant of claim 3, wherein the plurality of the struts forming the proximal region further includes a proximal second row of struts, and wherein the proximal second row of struts includes between 36 and 68 struts.

5. The occlusive implant of claim 4, wherein the plurality of the struts forming the distal region further includes a distal second row of struts, and wherein the distal second row of struts includes between 36 and 68 struts.

6. The occlusive implant of claim 5, wherein the plurality of the struts forming the proximal region further includes a proximal third row of struts, and wherein the proximal third row of struts includes between 36 and 68 struts.

7. The occlusive implant of claim 6, wherein the plurality of the struts forming the distal region further includes a distal third row of struts, and wherein the distal third row of struts includes between 36 and 68 struts.

8. The occlusive implant of claim 7, wherein the plurality of the struts forming the proximal region further includes a proximal fourth row of struts, and wherein the proximal fourth row of struts includes between 36 and 68 struts.

9. The occlusive implant of claim 8, wherein the plurality of the struts forming the distal region further includes a distal fourth row of struts, and wherein the distal fourth row of struts includes between 36 and 68 struts.

10. The occlusive implant of claim 9, wherein the plurality of the struts forming the proximal region further includes a proximal fifth row of struts, and wherein the proximal fifth row of struts includes between 36 and 68 struts.

11. The occlusive implant of claim 10, wherein the plurality of the struts forming the distal region further includes a distal fifth row of struts, and wherein the distal fifth row of struts includes between 36 and 68 struts.

12. The occlusive implant of claim 11, wherein a strut from the plurality of the struts forming the proximal region is connected to a strut from the plurality of the struts forming the engagement region at a proximal engagement node, and wherein the proximal engagement node includes an extrados region, an intrados region or both an extrados region and an intrados region.

13. The occlusive implant of claim 12, wherein a strut from the plurality of the struts forming the distal region is connected to a strut from the plurality of the struts forming the engagement region at a distal engagement node, and wherein the distal engagement node includes an extrados region, an intrados region or both an extrados region and an intrados region.

14. The occlusive implant of claim 1, wherein each strut of the proximal first row of struts and each strut of the distal first row of struts includes a width between 0.0025 inches and 0.020 inches and a thickness between 0.004 inches and 0.010 inches.

15. The occlusive implant of claim 14, wherein the ratio of the width to the thickness of each strut of the proximal first row of struts and each strut of the distal first row of struts is less than 1.0.

16. The occlusive implant of claim 1, wherein the proximal first row of struts includes a first strut and a second strut, and wherein the first strut and the second strut diverge from a node of the row of first connecting nodes to define an angle between 40 degrees and 80 degrees.

17. The occlusive implant of claim 1, further comprising an occlusive member disposed along an outer surface of the expandable framework.

18. An occlusive implant, comprising:
a distal hub;
a proximal hub; and
an expandable framework including a plurality of struts spaced around a central longitudinal axis of the occlusive implant and forming a distal region;
wherein the plurality of the struts forming the distal region includes a distal first row of struts coupled to a row of connecting nodes;
wherein the row of connecting nodes includes a first connecting node, a second connecting node and a third connecting node;
wherein the first connecting node has a first node length;
wherein the second connecting node has a second node length the same as the first node length;
wherein the third connecting node has a third node length different from the first node length;
wherein the third connecting node is disposed between the first connecting node and the second connecting node.

19. The occlusive implant of claim 18, wherein at least one node of the row of connecting nodes includes an extrados region, an intrados region or both an extrados region and an intrados region

20. An occlusive implant, comprising:
an expandable framework configured to shift between a collapsed configuration and an expanded configuration;
wherein the expandable framework includes a plurality of struts spaced around a central longitudinal axis of the expandable implant and forming a distal region, and a plurality of struts spaced around the central longitudinal axis of the expandable implant and forming a proximal region;
wherein the plurality of struts forming the distal region are configured to shift between a collapsed configuration and an expanded configuration;
wherein the plurality of struts forming the proximal region are configured to shift between a collapsed configuration and an expanded configuration;
wherein the plurality of struts forming the distal region are configured to maintain the expanded configuration when the struts forming the proximal region are in a partially expanded configuration; and
wherein the plurality of struts forming the proximal region are configured to maintain the expanded configuration when the struts forming the distal region are in a partially expanded configuration.
